(19) United States
(12) Patent Application Publication
(10) Pub. No.: US 2024/0131184 A1
(43) Pub. Date: Apr. 25, 2024

(54) COMPOSITIONS AND METHODS FOR DELIVERING NUCLEIC ACIDS TO CELLS

(71) Applicant: University of Maryland, Baltimore, Baltimore, MD (US)

(72) Inventors: Ryan Pearson, Baltimore, MD (US); Atanu Chakraborty, Baltimore, MD (US)

(21) Appl. No.: 17/767,783

(22) PCT Filed: Oct. 12, 2020
(86) PCT No.: PCT/US2020/055272

§ 371 Date: Apr. 8, 2022

Related U.S. Application Data
(60) Provisional application No. 62/913,430, filed on Oct. 10, 2019.

(51) Int. Cl. A61K 48/00; A61K 9/51; A61K 47/59; A61K 47/69; C12N 15/88

(52) U.S. Cl. CPC A61K 48/0041; A61K 9/5146; A61K 47/59; A61K 47/6933; C12N 15/88

(57) ABSTRACT

The present invention provides a non-viral polyplex particle for delivering nucleic acid to cells, comprising an effective amount of polyethylenimine complexed with an effective amount of the nucleic acid; and an effective amount of an anionic biomaterial that envelops the complexed acetylated polyethylenimine and nucleic acid.

### CROSS REFERENCE TO RELATED APPLICATIONS

This application claims the benefit of U.S. Provisional Appl. No. 62/913,430, filed Oct. 10, 2019, the contents of which are hereby incorporated by reference in their entirety.

### FIELD OF THE INVENTION

The field of the invention relates generally to compositions and methods for delivering nucleic acids to cells.

### BACKGROUND

The delivery of nucleic acids to lymphocytes has the potential to improve therapeutic outcomes through correction of genetic aberrations and development of next generation cell-based therapies (Pearson et al., Adv Drug Deliv Rev 2017, 114, 240-255). Recently, significant efforts have been focused on achieving in situ gene delivery to eliminate the need for time and labor intensive ex vivo immune cell manipulations (Smith et al., Nat Nanotechnol 2017, 12, 813-820.; Zhang et al., Nat Commun 2019, 10, 3974). Traditional methods for gene delivery rely heavily on physical, viral, or chemical approaches. Physical methods including electroporation, microinjection, ultrasound, or other hydrodynamic methods show high transfection efficiency yet suffer from high toxicity and are not amenable to cell-specific targeting, which hinders their potential in vivo applicability. Viral vectors are highly efficient, but the use of live viruses is limited by their potential mutagenicity and immunogenicity posing concerns for safety in clinical translation (Colella et al., Mol Ther Methods Clin Dev 2018, 8, 87-104.; Shirley et al., Mol Ther 2020, 28, 709-722.; Hacein-Bey-Abina et al., New England Journal of Medicine 2010, 363, 355-364). Chemical methods for gene delivery including calcium phosphate, lipids, cationic polymers, and others have the potential to overcome many of these limitations, particularly regarding toxicity and targetability in vivo.

Non-viral gene delivery has been routinely investigated for the delivery of various nucleic acid-based cargoes including plasmid DNA (pDNA), DNA oligonucleotides, mRNA, non-coding RNA species (i.e., siRNA, shRNA, lncRNA), Clustered Regularly Interspaced Short Palindromic Repeats/CRISPR-associated Protein 9 (CRISPR-Cas9) to alter the expression of target genes and translation of their gene products (Smith et al., Nat Nanotechnol 2017, 12, 813-820; Zhang et al., Nat Commun 2019, 10, 3974; Wan et al., J Control Release 2020, 322, 236-247; Moradian et al., Sci Rep 2020, 10, 4181; Hou et al., Nature Nanotechnology 2020, 15, 41-46; Billingsley et al., Nano Lett 2020, 20, 1578-1589; Ryu et al., Nanomedicine 2018, 14, 2095-2102; McKinlay et al., Proc Natl Acad Sci USA 2018, 115, E5859-E5866; Whitehead et al., Nat Commun 2014, 5, 4277). Cationic polymers like polyethylenimine (PEI), poly(β-amino ester) (PBAE), poly(L-lysine) (PLL), polyamidoamine (PAMAM) dendrimers, and others have shown variable abilities to efficiently transfect lymphocytes. Several groups have developed approaches for high-throughput synthesis of carriers with the objective to optimize lymphocyte transfection and mitigate toxicity using cationic polymers (such as PBAE and others) or lipid nanoparticles (LNPs) (Hou et al., Nature Nanotechnology 2020, 15, 41-46.; Billingsley et al., Nano Lett 2020, 20, 1578-1589; Whitehead et al., Nat Commun 2014, 5, 4277; Bishop et al., Journal of Controlled Release 2015, 219, 488-499; Patel et al., Nat Commun 2020, 11, 983). However, because of the wide variety of materials tested, it is difficult to draw direct relationships between studies based on the physicochemical properties of the materials to predict effectiveness of gene delivery.

PEI is one of the most effective off-the-shelf polymers used for gene delivery, yet it is associated with toxicity at high concentrations through cell membrane damage and apoptosis (Moghimi et al., Mol Ther 2005, 11, 990-5.; Choi et al., Drug Chem Toxicol 2010, 33, 357-66). Strategies to reduce the inherent toxicity of PEI by modification of its chemical properties such as using low molecular weight PEI, functionalization of PEI (i.e., acetylation, PEGylation, etc.), and PEI encapsulation have yielded significant improvements (Sunogrot et al., Bioconjugate Chemistry 2011, 22, 466-474.; Fitzsimmons et al., Acta Biomaterialia 2012, 8, 3941-3955.; Forres et al., Pharm Res 2004, 21, 365-71). PEI-based polyplexes are internalized via endosomal/lysosomal trafficking and escape from vesicles by the “proton-sponge” mechanism (Forres et al., Pharm Res 2004, 21, 365-71.; Kichler et al., J Gene Med 2001, 3, 135-44.; Godbey et al., Proc Natl Acad Sci USA 1999, 96, 5177-81.; Forrest et al., Mol Ther 2002, 6, 57-66). Once released into the cytosol, DNA must dissociate from PEI, be transported to the nucleus for gene transcription, and then shuttled back to the cytoplasm for translation to protein. To limit the toxicity associated with PEI and to increase the ability of polyplexes to unpackage DNA in the cells, acetylation of PEI (Ac-PEI) was evaluated. Ac-PEI modified the proton buffering capacity, increased the ability for polyplexes to unpackage cargos in the cell, and reduced the toxicity associated with unmodified PEI (Forres et al., Pharm Res 2004, 21, 365-71.; Gabrielson et al., Biomacromolecules 2006, 7, 2427-2435). However, only limited effectiveness has been demonstrated for PEI and Ac-PEI to transfect adaptive immune cells (Olden et al., J Control Release 2018, 282, 140-147.; Kircheis et al., Gene Ther 1997, 4, 409-18), potentially due to reduced proliferative capacity of resting immune cells and additional defense mechanisms within immune cells such as pattern recognition receptors.

An important challenge to overcome for efficient non-viral gene delivery is the biomolecule corona. The biomolecule corona forms due to the adsorption of serum proteins to the surface of nanoparticles, which can negatively affect the formation of specific cellular interactions as well as reduce transfection efficiency (Pearson et al., Front Chem 2014, 2, doi: 10.3389/fchem.2014.00108.; Al-Dosari et al., AAPS J 2009, 11, 671-81; Salvati et al., Nature Nanotechnology 2013, 8, 137-143). Several groups have attempted to overcome the negative contribution of serum on transfection efficiency. Olden et al. evaluated the transfection efficiency of Jurkat cells in serum-free and serum-containing media using comb- and sunflower-type poly(2-dimethylaminoethyl methacrylate) (pDMAEMA) cationic polymer architectures (Olden et al., J Control Release 2018, 282, 140-147). Serum-free transfection conditions of Jurkat cells using either polymer type resulted in approximately 30% and 10% gene expression, respectively. However, in serum containing medium, the efficiencies were significantly reduced to approximately 7% and 6%, respectively. Notably, the use of PEI as a control did not result in any measurable transfection in either condition. Another group focused on transfection of hard-to-transfect lymphoma/leukemia cells (Zhao et al., J Control Release 2012, 159, 104-10). Using poly(β-amino ester) (PBAE) polymers in vitro, gene expression was found to be limited in serum-free conditions. However, pre-treatment of cells with polybrene prior to the addition of PBAE polyplexes was necessary to yield significant improvements in gene expression (up to 32%), an 8-fold increase over that mediated by Lipofectamine®. The need to pre-treat cells prior to transfection is expected to hinder the in vivo applicability of these PBAE polymers. Many of these strategies have improved upon issues related to limited endocytosis, protein expression, and low cell viability; however, the relative complexity in formulation design or treatment schedules, in addition to reduced gene expression in the presence of serum may limit their utility for in vivo applications and subsequent future development. Thus, a critical need remains to establish simple and effective methods to transfect pDNA into lymphocytes.

Polyelectrolyte enveloping of cationic polyplexes enables the surface chemistry of substrates to be modified through electrostatic absorption with the purpose of decreasing toxicity, reducing non-specific binding interactions, enabling enhanced cell targeting, and co-delivery of multiple therapeutic agents (Bishop et al., J Biomed Mater Res A 2016, 104, 707-713; Shmueli et al., Expert Opinion on Drug Delivery 2010, 7, 535-550; Hammond et al., Materials Today 2012, 15, 196-206). Anionic peptides have been used to coat the surface of cationic polyplexes to reduce the non-specific interactions for a targeting application using HUVECs (Green et al., Nano Letters 2007, 7, 874-879). Gene delivery to immune cells was achieved in vivo using PBAE polyplexes coated with poly(glutamic acid) for transfection of T cells or tumor-associated macrophages (Smith et al., Nat Nanotechnol 2017, 12, 813-820.; Zhang et al., Nat Commun 2019, 10, 3974). More recently, modification of nanoparticle surfaces gave rise to controlled cellular interactions and specificity to ovarian cancer, demonstrating the crucial role played by each polyelectrolyte used in the formulation (Correa et al., ACS Nano 2020, 14, 2224-2237).

There is a need for new particle compositions capable of delivering nucleic acids to cells in vivo, particularly to cells of the immune system.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

Here, the present inventors describe the development of immunoplexes (IPs) that consist of an inner Ac-PEI/pDNA polyplex enveloped within an anionic poly(ethylene-alt-maleic acid) (PEMA) polyelectrolyte layer for the serum-independent transfection of innate and adaptive immune cells. IPs were prepared and screened for various physical and biochemical properties and pDNA transfection efficiency was determined in RAW 264.7 macrophages, DC2.4 dendritic cells, and human Jurkat T cells. Cellular interactions of IPs were controllable through modulation of PEMA content and correlated with levels of gene expression. By incorporating pDNA into IPs at unusually high N/P ratios, IP stability in the presence of serum was greatly improved, PEMA enveloping improved pDNA unpackaging from IPs, and mitigated toxicity associated with Ac-PEI delivery at high N/P ratios. The ability to specifically control the cellular interactions of IPs highlights their potential use as a modular and targetable gene delivery platform to achieve high levels of cell-specific transfection in vivo. These results support IPs as a simple, modular, serum-independent, and highly effective non-viral gene delivery platform to efficiently transfect cells of the innate and adaptive immune system.

In one aspect, the invention provides a non-viral polyplex particle for delivering nucleic acid to cells, comprising
i) an effective amount of polyethylenimine complexed with an effective amount of the nucleic acid; and
ii) an effective amount of an anionic biomaterial that envelops the complexed polyethylenimine and nucleic acid.

In some embodiments, the polyethylenimine is acetylated. In some embodiments, the polyethylenimine is branched. In some embodiments, the polyethylenimine is linear.

In some embodiments, the non-viral polyplex particle has an N/P ratio of at least 7.5. In some embodiments, the non-viral polyplex particle has an N/P ratio of at least 15. In some embodiments, the non-viral polyplex particle has an N/P ratio of at least 30.

In another aspect, the invention provides a non-viral polyplex particle for delivering a nucleic acid to cells, comprising
i) an effective amount of an amine containing cationic biomaterial complexed with an effective amount of the nucleic acid; and
ii) an effective amount of an anionic biomaterial that envelops the complex of amine containing cationic biomaterial and nucleic acid, wherein the non-viral polyplex particle has an N/P ratio of at least 15.

In some embodiments, the amine containing cationic biomaterial is selected from the group consisting of polylysine, polyethylenimine, chitosan, polyamidoamine dendrimers, polyhistine, poly(beta-amino esters), poly(2-dimethylaminoethyl methacrylate), poly(2-[(2-aminoethyl)amino] ethyl aspartamide), poly(2-aminoethyl ethylene phosphate), spermine, spermidine, a cationic lipid and combinations thereof.

In some embodiments, the cationic lipid is selected from the group consisting of DOPE, DOTAP, DOGS, DOSPER and combinations thereof.

In some embodiments, the anionic biomaterial is selected from the group consisting of poly(glutamic acid), poly(aspartic acid), poly(acrylic acid), hyaluronic acid, poly(methyl vinyl ether-alt-maleic acid), poly(isobutylene-alt-maleic acid), poly(ethylene-alt-maleic acid), poly(ethylene-alt-maleic anhydride), and combinations thereof. In some embodiments, the the anionic biomaterial is poly(ethylene-alt-maleic acid).

In some embodiments, the anionic biomaterial is present at a weight percent of about 0.1% to about 67% relative to the weight of the particle. In some embodiments, the anionic biomaterial is present at a weight percent of about 5% to about 35%.

In some embodiments, the amine containing cationic biomaterial is polyethylenimine. In some embodiments, the polyethylenimine is linear or branched. In some embodiments, the polyethylenimine is acetylated.

In some embodiments, the percentage acetylation of polyethylenimine is from about 10% to about 60%. In some embodiments, the percentage acetylation of polyethylenimine is from about 20% to about 50%. In some embodiments, the percentage acetylation of polyethylenimine is from about 20% to about 25%. In some embodiments, the percentage acetylation of polyethylenimine is about 25%.

In some embodiments, the non-viral polyplex particle has a zeta potential of at least about +10. In some embodiments, the non-viral polyplex particle has a zeta potential of between about +25 to about +30. In some embodiments, the non-viral polyplex particle has a zeta potential less than about +50.

In some embodiments, the non-viral polyplex particle further comprises a targeting moiety that enables delivery of the nucleic acids to a target cell, wherein the targeting moiety binds to the surface of the target cell, wherein the non-viral polyplex particle is internalized by the target cell by receptor-mediated endocytosis.

In some embodiments, the targeting moiety is selected from the group consisting of a protein, a cell adhesion molecule, an antibody, a peptide, a sugar, a small molecule, and any combination thereof.

In some embodiments, the targeting moiety comprises a single chain antibody. In some embodiments, the targeting moiety comprises a single chain (scFv) variable fragment antibody. In some embodiments, the targeting moiety comprises a Fab fragment.

In some embodiments, the targeting moiety binds to a cell surface molecule or complex selected from the group consisting of mannose receptor (CD206), folic acid receptor, Ly6G, Ly6C and CD3.

In some embodiments, the nucleic acid is selected from the group consisting of DNA, messenger RNA, small interfering RNA (siRNA), short hairpin RNA (shRNA), antisense oligonucleotides (ODN) and combination thereof. In some embodiments, the DNA comprises plasmid DNA.

In another aspect, the invention provides a method of delivering a nucleic acid to a target cell, comprising administering an effective amount of the non-viral polyplex particle as described herein.

In another aspect, the invention provides a method of delivering a nucleic acid to a target cell in a subject, comprising administering an effective amount of the non-viral polyplex particle as described herein.

In some embodiments, the target cell is an immune cell. In some embodiments, the immune cell is selected from neutrophils, bone marrow cells, bone marrow stem cells, NK cells, dendritic cells, monocytes, B lymphocytes, macrophages, and T lymphocytes.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. Synthesis and characterization of acetylated PEI (Ac-PEI). A) Synthesis scheme for acetylated PEI. B) Representative ¹H-NMR spectra for PEI and Ac-PEI in D₂O. The peak at 1.7-1.8 ppm is due to acetylated primary amines whereas the peak at 1.8-1.9 ppm is from acetylated secondary amines of PEI. C) Quantification of PEI degree of acetylation and molecular weight determined by ¹H-NMR for various modified PEI polymers.

FIG. 2. Ac-PEI polyplex formation and characterization. A) Schematic representation of polyplex formation using acetylated PEI (Ac-PEI) and plasmid DNA (pDNA). B) Agarose gel electrophoresis showing complex formation of various Ac-PEI polymers with GFP plasmid at different N/P ratios. C) GFP expression in RAW 264.7 macrophages using AC-PEI/GFP polyplexes prepared at N/P ratio 20. Ac₂₀-PEI shows higher GFP signals compared to Ac₄₀-PEI and Ac₆₀-PEI. Representative images of at least n=3 experiments.

FIG. 3. Serum-dependent transfection efficiency and toxicity of Ac-PEI polyplexes. Transfection efficiency of Ac₂₀-PEI (Ac-PEI) polyplexes at various N/P ratios in RAW 264.7 (A) and Jurkat cells (B) determined by flow cytometry. Median Fluorescence Intensity of GFP expression for RAW 264.7 (C) and Jurkat cells (D). Cell viability determined by MTS assay for RAW 264.7 (E) and Jurkat cells (F). Cells were incubated in serum-free or serum-containing medium for 4 hrs with polyplexes, washed, and incubated for 24 hrs prior to analysis. Data are representative of n=3 experiments. Statistical differences between groups were determined by performing a one-way ANOVA and Tukey's post hoc test (p<0.05). Error bars represent SD

FIG. 4. Preparation, characterization, and stability assessment of immunoplexes (IPs). A) Schematic representation of enveloping Ac-PEI/GFP polyplexes with various wt. % of PEMA to form IPs. B, C) Hydrodynamic size and zeta potential of IPs, respectively. Agarose gel electrophoresis to assess the stability of Ac-PEI/GFP polyplexes following PEMA enveloping at N/P 30 in the absence (D) or presence (E) of physiologically-relevant concentration serum (55% v/v). IPs are stable after enveloping with PEMA and in presence of serum as no DNA band is observed. PEMA (poly(ethylene-alt-maleic acid)). n=3 for each experiment. Statistical differences between groups were determined by performing a one-way ANOVA and Tukey's post hoc test (p<0.05). All bars in panel B are significantly different from each other. Error bars represent SD.

FIG. 5. Serum- and PEMA enveloping-dependent transfection and toxicity of IPs. Transfection efficiency of IPs prepared with various wt. % of PEMA (10% to 50%) determined by flow cytometry in RAW 264.7 (A) and Jurkat cells (B). Median Fluorescence Intensity of GFP expression for RAW 264.7 (C) and Jurkat cells (D). Cell viability determined by MTS assay for RAW 264.7 (E) and Jurkat cells (F). Cells were incubated in serum-free or serum-containing medium for 4 hrs with polyplexes, washed, and incubated for 24 hrs prior to analysis. Data are representative of n=3 experiments. Statistical differences between groups were determined by performing a one-way ANOVA and Tukey's post hoc test (p<0.05). Error bars represent SD.

FIG. 6. Persistence of GFP expression in RAW 264.7 macrophages. A) Fluorescence micrographs of RAW 264.7 macrophages transfected in serum-containing medium with various IPs enveloped with various wt. % of PEMA. Images are representative of at least n=3 experiments. Cells were incubated in serum-containing medium for 4 hrs with IPs, excess IPs were washed away, and images were acquired at various time points. B) Transfection efficiency of IPs, 24 hr post cell treatment. C) Fold-change enhancements in transfection efficiency relative to Ac-PEI30 for IPs. IP₃₀-10 displayed significantly longer and higher levels of transfection compared to Ac-PEI30 polyplexes. Data are representative of n=3 experiments and as mean+/−SD.

FIG. 7. ¹H-NMR of various PEI polymers used in this study. A) PEI. B) Ac₂₀-PEI. C) Ac₄₀-PEI. D) Ac₆₀-PEI. Spectra were measured in D₂O and calibrated to the solvent signal at 4.8 ppm. Panels A and B are duplicated from FIG. 1.

FIG. 8. Experimental setup for transfections. A) Schematic of transfection experiment for immunoplexes. B) Representative gating strategy for flow cytometry for untreated control and Ac-PEI30 in serum containing medium.

FIG. 9. Controlled cellular interactions and transfection efficiency of IPs. Fluorescence micrographs of RAW 264.7 (A) or Jurkat cells (B) transfected in serum-containing medium with IPs enveloped with various wt. % of PEMA. Ac-PEI-RHO was used in IPs to establish the relationship between cellular interactions and transfection efficiency. Cells were incubated in serum-containing medium for 4 hrs with IPs, excess IPs were washed away, and images were acquired 24 hr later. Images are representative of at least n=3 experiments. White arrows show areas of colocalization of GFP/RHO signals.

FIG. 10. Serum- and PEMA enveloping-dependent transfection of IPs in DC2.4 cells. Percentage of transfection (A) and Median Fluorescence Intensity of GFP expression (B) of IPs prepared with various wt. % of PEMA (10% to 50%) determined by flow cytometry in DC2.4 cell. Cells were incubated in serum-free or serum-containing medium for 4 hrs with polyplexes, washed, and incubated for 24 hrs prior to analysis. Data are representative of n=3 experiments. Statistical differences between groups were determined by performing a one-way ANOVA and Tukey's post hoc test (p<0.05). Error bars represent SD.

FIG. 11. 1HNMR characterization of a) poly n-Boc serinol and b) Poly serinol in DMSO d⁶. The peak around 1.4 in a) indicates the presence of Boc protecting group which was disappeared after reaction with TFA (b).

FIG. 12. 1HNMR spectra of poly diethanolamine in D2O.

FIG. 13. Agarose gel electrophoresis of polyplexes prepared with two different type of polymers (polyserinol and polydiethanolamine respectively) and GFP plasmid. It shows that polydiethanolamine can form more stable polyplexes with plasmid than polyserinol.

FIG. 14. GFP expression in RAW 264.7 macrophage cells using polyplex^{diethanolamine}.

### DETAILED DESCRIPTION

As described herein, genetic engineering of innate and adaptive immune cells represents a potential solution to treat numerous immune-mediated pathologies. Current immune engineering methods to introduce nucleic acids into cells with high efficiency rely on physical mechanisms such as electroporation, viral vectors, or other chemical methods. Gene delivery using non-viral nanoparticles offers significant flexibility in biomaterial design to tune critical parameters such as nano-bio interactions, transfection efficiency, and toxicity profiles. However, their clinical utility has been limited due to complex synthetic procedures, high toxicity at increased polymer (nitrogen, N) to DNA ratios (phosphate, P) (N/P ratios), poor transfection efficiency and nanoparticle stability in the presence of serum, and short-term gene expression. Here, we describe the development of a simple, polymer-based non-viral gene delivery platform based on simple modifications of polyethylenimine (PEI) that displays potent and serum-independent transfection of innate and adaptive immune cells. Cationic acetylated PEI (Ac-PEI) was synthesized and complexed with plasmid DNA (pDNA) followed by enveloping with an anionic polyelectrolyte layer of poly(ethylene-alt-maleic acid) (PEMA) to form immunoplexes (IPs). Cellular interactions and gene expression could be precisely controlled in murine RAW 264.7 macrophages, murine DC2.4 dendritic cells, and human Jurkat T cells by altering the levels of PEMA envelopment, thus providing a strategy to engineer specific cell targeting into the IP platform. Optimally formulated IPs for immune cell transfection in the presence of serum utilized high N/P ratios to enable high stability, displayed reduced toxicity, high gene expression, and a lengthened duration of gene expression (>3 days) compared to non-enveloped controls. These results demonstrate the potential of engineered IPs to serve as simple, modular, targetable, and efficient non-viral gene delivery platform to efficiently alter gene expression within cells of the immune system.

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

In some embodiments, the practice of the present invention employs various techniques of molecular biology (including recombinant techniques), microbiology, cell biology, biochemistry and immunology. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “an antibody” includes a plurality of antibodies, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

The term “nanoparticle” as used herein refers to a particle having a size from about 1 nm to about 1000 nm.

The term “particle” as used herein refers to a composition having a size from about 1 nm to about 1000 m.

The term “particle size” as used herein refers to the median size in a distribution of particles. The median size is determined from the average linear dimension of individual particles, for example, the diameter of a spherical particle. Size may be determined by any number of methods in the art, including dynamic light scattering (DLS) and transmission electron microscopy (TEM) techniques.

References to a composition described and disclosed herein are considered to include the free acid, the free base, and all addition salts. The compositions may also form inner salts or zwitterions when a free carboxy and a basic amino group are present concurrently. The term “pharmaceutically acceptable salt” refers to salts which possess toxicity profiles within a range that affords utility in pharmaceutical applications. In general the useful properties of the compositions described herein do not depend on whether the composition is or is not in a salt form, so unless clearly indicated otherwise (such as specifying that the composition should be in “free base” or “free acid” form), reference in the specification to a composition should be understood as including salt forms of the composition, whether or not this is explicitly stated. Preparation and selection of suitable salt forms is described in Stahl et al., Handbook of Pharmaceutical Salts: Properties, Selection, and Use, Wiley-VCH 2002.

When in the solid state, the compositions described herein and salts thereof may occur in various forms and may, e.g., take the form of solvates, including hydrates. In general, the useful properties of the compositions described herein do not depend on whether the composition or salt thereof is or is in a particular solid state form, such as a polymorph or solvate, so unless clearly indicated otherwise reference in the specification to compositions and salts should be understood as encompassing any solid state form of the composition, whether or not this is explicitly stated.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented.

The terms “modulation” and “modulate” as used herein refer to a change or an alteration in a biological activity. Modulation includes, but is not limited to, stimulating an activity or inhibiting an activity. Modulation may be an increase or a decrease in activity, a change in binding characteristics, or any other change in the biological, functional, or immunological properties associated with the activity of a protein, a pathway, a system, or other biological targets of interest.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments, dual affinity retargeting antibodies (DART)), single chain Fv (scFv) mutants, multispecific antibodies such as bispecific and trispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

In some embodiments, an antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations.

The terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antigen binding fragment” or antibody fragment refers to a portion of an intact antibody and comprises the antigenic determining variable regions of an intact antibody. Examples of antigen binding fragment include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g. murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)).

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommie, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cysteine 23 (Ist-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles (Ruiz, M. and Lefranc, M.-P., Immunogenetics, 53, 857-883 (2002)/Kaas, Q. and Lefranc, M.-P., Current Bioinformatics, 2, 21-30 (2007)), and in 3D structures in IMGT/3Dstructure-DB (Kaas, Q., Ruiz, M. and Lefranc, M.-P., T cell receptor and MHC structural data. Nucl. Acids. Res., 32, D208-D210 (2004)).

In some embodiments, CDRs are determined based on cross-species sequence variability (i.e., Kabat et al. Sequences of Proteins of Immunological Interest, (5th ed., 1991, National Institutes of Health, Bethesda Md.)). In some embodiments, CDRs are determined based on crystallographic studies of antigen-antibody complexes (Al-lazikani et al (1997) J. Molec. Biol. 273:927-948)). In addition, combinations of these two approaches can be used to determine CDRs. In some embodiments, the CDRs are determined based on AHo (Honegger and Pluckthun, J. Mol. Biol. 309(3):657-670; 2001). In some embodiments, CDRs are determined based on the IMGT system.

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

An “intact” antibody is one that comprises an antigen-binding site as well as a C_{L} and at least heavy chain constant domains, C_{H1}, C_{H2} and C_{H3}. The constant domains may be native sequence constant domains (e.g., human native sequence constant domains) or amino acid sequence variants thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

The antibodies that can be useful herein also include antibodies in which a portion of the heavy and/or light chain is identical with or homologous to corresponding sequences in antibodies belonging to a particular antibody class or subclass, while the remainder of the chain(s) is identical with or homologous to corresponding sequences in antibodies derived from another species or belonging to another antibody class or subclass, as well as fragments of such antibodies.

In some embodiments, the antibody comprises variable region antigen-binding sequences derived from human antibodies (e.g., CDRs) and containing one or more sequences derived from a non-human antibody, e.g., an FR or C region sequence. In some embodiments, the antibody includes those comprising a human variable region antigen binding sequence of one antibody class or subclass and another sequence, e.g., FR or C region sequence, derived from another antibody class or subclass.

In some embodiments, chimeric antibodies may comprise residues that are not found in the recipient antibody or in the donor antibody. In some embodiments, modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

The term “binding” refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Binding partner” or “ligand” refers to a molecule that can undergo specific binding with a particular molecule. “Biological binding” defines a type of interaction that occurs between pairs of molecules including proteins, peptides, nucleic acids, glycoproteins, carbohydrates, or endogenous small molecules. “Specific binding” refers to molecules, such as polynucleotides, that are able to bind to or recognize a binding partner (or a limited number of binding partners) to a substantially higher degree than to other, similar biological entities.

As used herein, the term “specifically binds” refers to the binding of a molecule, e.g., a drug, to a protein molecule, while not significantly binding to other protein molecules. In some embodiments, an antibody “specifically binds” to an protein molecule with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more). As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “and/or” as used in a phrase such as “A and/or B” herein is intended to include both A and B; A or B; A (alone); and B (alone). Likewise, the term “and/or” as used in a phrase such as “A, B. and/or C” is intended to encompass each of the following embodiments: A. B, and C; A, B. or C; A or C; A or B; B or C; A and C; A and B; B and C; A (alone), B (alone); and C (alone).

### Non-Viral Polyplex Particles and Methods of Use

In one embodiment, the invention provides a non-viral polyplex particle for delivering nucleic acid to cells, comprising
i) an effective amount of polyethylenimine complexed with an effective amount of the nucleic acid; and
ii) an effective amount of an anionic biomaterial that envelops the complexed polyethylenimine and nucleic acid.

In another embodiment, the invention provides a non-viral polyplex particle for delivering a nucleic acid to cells, comprising
i) an effective amount of an amine containing cationic biomaterial complexed with an effective amount of the nucleic acid; and
ii) an effective amount of an anionic biomaterial that envelops the complex of amine containing cationic biomaterial and nucleic acid,

wherein the non-viral polyplex particle has an N/P ratio of at least 15.

In some embodiments, the particle further comprises one or more additional layers of polyethylenimine (or other cationic biomaterial) and one or more additional layers of anionic biomaterial. In some embodiments, such layers can be added sequentially using a layer-by-layer (LbL) approach. For example, an additional layer comprising polyethylenimine (or cationic biomaterial) can coated onto the anionic biomaterial that envelops the complexed polyethylenimine (or other cationic biomaterial) and nucleic acid, followed by coating of an additional layer of anionic biomaterial.

In another embodiment, the invention provides a method of delivering a nucleic acid to a target cell, comprising administering an effective amount of the non-viral polyplex particles as described herein.

In another embodiment, the invention provides a method of delivering a nucleic acid to a target cell in a subject, comprising administering an effective amount of the non-viral polyplex particles as described herein.

The methods can be performed in vitro, in vivo or ex vivo. When performed in vitro or ex vivo, the methods can be performed in the presence or absence of serum. Advantageously, the non-viral polyplex particles can be efficiently delivered and taken up by cells in vivo, in the presence of serum.

In some embodiments, the invention provides for a method of treating a disease or condition in a subject in need thereof, comprising administering to the subject a therapeutically effective amount of a composition as described herein.

The compositions of the disclosure can be used in any method of treating a disease or condition beneficially treated by administration of a nucleic acid in a subject.

The term “subject” refers to any animal (e.g., a mammal), including, but not limited to, humans, non-human primates, canines, felines, rabbits, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of an agent described herein, an antibody, a polypeptide, a polynucleotide, an amine containing cationic biomaterial, an anionic biomaterial, a small organic molecule, or other drug effective to deliver nucleic acids to cells, or otherwise “prevent” or “treat” a disease or disorder in a subject such as, a mammal.

The cell that can be delivered the nucleic acid by the polyplex particle is not particularly limiting. The cell becomes transfected upon uptake of the polyplex particle. The term “transfect” refers to introduction of a molecule such as a nucleic acid (plasmid) into a cell. A cell has been “transfected” when exogenous nucleic acid has been introduced inside the cell membrane. Accordingly, a “transfected cell” is a cell into which a “nucleic acid” or “polynucleotide” has been introduced, or a progeny thereof in which an exogenous nucleic acid has been introduced. In particular embodiments, a “transfected cell” cell (e.g., in a mammal, such as a cell or tissue or organ cell) is a genetic change in a cell following incorporation of an exogenous molecule, for example, a nucleic acid (e.g., a transgene). A “transfected” cell(s) can be propagated and the introduced nucleic acid transcribed and/or protein expressed.

In a “transfected” cell, the nucleic acid (plasmid) may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extrachromosomally, or only transiently.

In some embodiments, the transfected cell is selected from a blood cell, a cancer cell, an immune cell (e.g., a macrophage cell), an epithelial cell (e.g., a skin cell), or a virus-infected cell.

In some embodiments, immune cells are transfected. In some embodiments, the immune cell is selected from neutrophils, bone marrow cells, bone marrow stem cells, NK cells, dendritic cells, monocytes, B lymphocytes, macrophages, and T lymphocytes.

In some embodiments, the transfected cell is a cancer cell. In some embodiments, the cancer cell is selected from a breast cancer cell, a colon cancer cell, a leukemia cell, a bone cancer cell, a lung cancer cell, a bladder cancer cell, a brain cancer cell, a bronchial cancer cell, a cervical cancer cell, a colorectal cancer cell, an endometrial cancer cell, an ependymoma cancer cell, a retinoblastoma cancer cell, a gallbladder cancer cell, a gastric cancer cell, a gastrointestinal cancer cell, a glioma cancer cell, a head and neck cancer cell, a heart cancer cell, a liver cancer cell, a pancreatic cancer cell, a melanoma cancer cell, a kidney cancer cell, a laryngeal cancer cell, a lip or oral cancer cell, a lymphoma cancer cell, a mesothioma cancer cell, a mouth cancer cell, a myeloma cancer cell, a nasopharyngeal cancer cell, a neuroblastoma cancer cell, an oropharyngeal cancer cell, an ovarian cancer cell, a thyroid cancer cell, a penile cancer cell, a pituitary cancer cell, a prostate cancer cell, a rectal cancer cell, a renal cancer cell, a salivary gland cancer cell, a sarcoma cancer cell, a skin cancer cell, a stomach cancer cell, a testicular cancer cell, a throat cancer cell, a uterine cancer cell, a vaginal cancer cell, and a vulvar cancer cell. For example, the cancer cell can be a lung cancer cell.

The polyplex particles and methods of the invention can be used to treat a cancer in a subject. Cancers include, but are not limited to, an adrenal cancer, a breast cancer, a colon cancer, a leukemia, a bile duct cancer, a bone cancer, a lung cancer (e.g., non-small cell lung cancer, small cell lung cancer, and lung carcinoid tumor), a bladder cancer, a brain cancer, a bronchial cancer, a cervical cancer, a colorectal cancer, an endometrial cancer, an ependymoma, a retinoblastoma, a gallbladder cancer, a gastric cancer, a gastrointestinal cancer, a glioma, a head and neck cancer, a heart cancer, a liver cancer, a pancreatic cancer, a melanoma, a kidney cancer, a laryngeal cancer, a lip or oral cancer, a lymphoma, a mesothioma, a mouth cancer, a myeloma, a nasopharyngeal cancer, a neuroblastoma, an oropharyngeal cancer, an ovarian cancer, a thyroid cancer, a penile cancer, a pituitary cancer, a prostate cancer, a rectal cancer, a renal cancer, a salivary gland cancer, a sarcoma, a skin cancer, a stomach cancer, a testicular cancer, a throat cancer, a uterine cancer, a vaginal cancer, and a vulvar cancer.

In some embodiments, the methods of the present invention can be used to treat an inflammatory disease or disorder, which can include sepsis, arthritis, multiple sclerosis, rheumatoid arthritis, psoriasis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis, Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, polymyalgia rheumatica, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, reperfusion injury, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis. Inflammatory diseases or conditions with an inflammatory component not triggered by autoimmunity are also included. See, for example, Tabas, I; Glass, C. K. Science 339 (6116): page 169 (2013).

In some embodiments, the methods enable reprogramming of a dysregulated immune response in a subject, for example, to improve cell migration, overcome significant cell death induced by over stimulation, increase pathogen clearance, or reduce apoptosis induction in T cells. In some embodiments, the methods deliver siRNA specifically to T cells targeting pro-apoptotic Bcl-2 family member Bim and Fas during T cell apoptosis in sepsis.

In some embodiments, the methods of the invention can be used to treat viral or bacterial infections. In some embodiments, the methods of the invention can be used to prevent or to treat a viral disease in a subject. In some embodiments, the viral disease is selected from the group consisting of: Adenovirus infections, Herpes virus infections (e.g., HSV-1, HSV-2 and varicella zooster virus infections), Papillomavirus infections (e.g., HPV-1, HPV-2, HPV-5, HPV-6, HPV-11, HPV-13, HPV-16, and HPV-18), Parvovirus infections, Polyomavirus infections, Poxvirus infections, Arbovirus infection, Arenavirus infections, Astrovirus infections, Birnavirus infections, Bunyavirus infections, Calicivirus infections, Coronavirus infections (e.g., SARS-CoV-2), Flavivirus infections, Hantavirus infections, Hepatitis virus infections (e.g., Hepatitis A, Hepatitis B, Hepatitis C, and Hepatitis D), Bornavirus infections, Filovirus infections, (e.g., Ebola virus, Marburg virus, and Cueva virus), Paramyxovirus infections (e.g., respiratory syncytial virus), and Rhabdovirus infections), Nidovirales infections, Orthomyxoviridae infections (e.g., influenza virus infections), Picornavirus infections (e.g., Enterovirus infections), Reovirus infections (e.g., Rotavirus infections), Retrovirus infections (e.g., lentivirus infections, e.g., HIV infections), and Togavirus infections (e.g., Rubivirus infections). In some embodiments, the nucleic acid (e.g., siRNA) prevents the replication of a virus causing disease. In some embodiments, the polyplex particles can function as vaccines by delivering nucleic acids encoding antigens to antigen presenting cells, such as dendritic cells. In some embodiments, the polyplex particle comprises a targeting moiety that targets uptake of the nucleic acid by antigen presenting cells, such as dendritic cells. In some embodiments, the nucleic acid encodes a polypeptide comprising a viral antigen or antigenic fragment.

In some embodiments, the methods of the invention can be used to treat a disease or a condition in need of enzyme (i.e., gene) replacement in a subject. For example, MPS disorders (mucopolysaccharidoses) are lysosomal storage diseases caused by the inability to produce specific enzymes, which in turn leads to an abnormal storage of mucopolysaccharides. In some embodiments, the disease in need of enzyme replacement is selected from the group consisting of: Gaucher disease, Fabry disease, Hurler syndrome (MPS I H), Scheie syndrome (MPS I S), Hurler-Scheie syndrome (MPS I H-S), Hunter syndrome (MPS II), Sanfilippo syndrome (e.g. Sanfilippo A (MPS III A), Sanfilippo B (MPS III B), Sanfilippo C (MPS III C), and Sanfilippo D (MPS III D)), Morquio syndrome (e.g. Morquio A (MPS IV A) and Morquio B (MPS IV B)), Maroteaux-Lamy syndrome (MPS VI), Sly syndrome (MPS VII), MPS IX (hyaluronidase deficiency), I-cell disease (ML II), Pseudo-Hurler polydystrophy (ML III), and Glycogen storage disease type II (Pompe disease). In some embodiments, the nucleic acid encodes a polypeptide useful to treat a disease in need of enzyme replacement. In some embodiments, the nucleic acid encodes a protein (e.g., an enzyme) that is deficient and/or less active in a subject suffering from a disease in need of enzyme replacement. In some embodiments, the protein comprises one or more proteins selected from the group consisting of: agalsidase beta, imiglucerase, velaglucerase alfa, taliglucerase, alglucosidase alfa, laronidase, idursulfase, and galsulfase.

In some embodiments, the particle size of the polyplex particle can be in a range from about 10 nm to about 10 m. In some embodiments, the size can be in a range from about 10 nm to about 1000 nm, from about 100 nm to about 950 nm, from about 150 nm to about 800 nm, and/or from about 200 nm to about 600 nm. For example, in some embodiments, the particle size can be about 100 nm, about 125 nm, about 150, about 175 nm, about 200 nm, about 2250 nm, about 250 nm, about 275 nm, about 300 nm, about 325 nm, about 350 nm, about 375 nm, about 400 nm, about 425 nm, about 450 nm, about 475 nm, about 500 nm, about 550 nm, about 600 nm, about 650 nm, about 700 nm, about 750 nm, about 800 nm, about 850 nm, about 900 nm, about 950 nm, or about 1000 nm.

In some embodiments, the polyplex particles present within a population, e.g., in a composition, can have substantially the same shape and/or size (i.e., they are “monodisperse”). For example, the particles can have a distribution such that no more than about 5% or about 10% of the particles have a diameter greater than about 10% greater than the average diameter of the particles, and in some cases, such that no more than about 8%, about 5%, about 3%, about 1%, about 0.3%, about 0.1%, about 0.03%, or about 0.01% have a diameter greater than about 10% greater than the average diameter of the particles.

In some embodiments, the diameter of no more than 25% of the particle varies from the mean particle diameter by more than 150%, 100%, 75%, 50%, 25%, 20%, 10%, or 5% of the mean particle diameter. It is often desirable to produce a population of particle that is relatively uniform in terms of size, shape, and/or composition so that most of the particles have similar properties. For example, at least 80%, at least 90%, or at least 95% of the particles produced using the methods described herein can have a diameter or greatest dimension that falls within 5%, 10%, or 20% of the average diameter or greatest dimension. In some embodiments, a population of particles can be heterogeneous with respect to size, shape, and/or composition.

In some embodiments, the amine containing cationic biomaterial is selected from the group consisting of polylysine, polyethylenimine, chitosan, polyamidoamine dendrimers, polyhistine, poly(beta-amino esters), poly(2-dimethylaminoethyl methacrylate), poly(2-[(2-aminoethyl)amino] ethyl aspartamide), poly(2-aminoethyl ethylene phosphate), spermine, spermidine, a cationic lipid and combinations thereof. In some embodiments, the cationic lipid is selected from the group consisting of DOPE, DOTAP, DOGS, DOSPER and combinations thereof.

In some embodiments, the amine containing cationic biomaterial is polyethylenimine (PEI). PEI is a cationic polymer and is able to form a stable complex with nucleic acid, referred to as a polyplex. Although not wishing to be bound by any theory, the polyplex is believed to be introduced into cells through endocytosis.

PEI can be linear PEI or branched PEI. PEI can be in a salt form or free base. In some embodiments, PEI is linear PEI, such as an optionally hydrolyzed linear PEI. The hydrolyzed PEI may be fully or partially hydrolyzed. Hydrolyzed linear PEI has a greater proportion of free (protonatable) nitrogen compared to non-hydrolyzed linear PEI, typically having at least 1-5% more free (protonatable) nitrogen compared to non-hydrolyzed linear PEI, more typically having 5-10% more free (protonatable) nitrogen compared to non-hydrolyzed linear PEI, or most typically having 10-15% more free (protonatable) nitrogen compared to non-hydrolyzed linear PEI.

In some embodiments, PEI can have a molecular weight in the range of about 4,000 to about 160,000 Da and/or in the range of about 2,500 to about 250,000 Da in free base form. In some embodiments, PEI can have a molecular weight of about 40,000 Da and/or about 25,000 Da in free base form. Specifically, linear PEI with a molecular weight of about 40,000 Da and/or about 25,000 Da in free base form. In addition, chemically modified linear PEI or branched PEI can be also used. PEI is commercially available (e.g., Polysciences, Inc., Warrington, Pa., USA).

In some embodiments, the PEI is acetylated. As provided herein, “percentage acetylation” refers to the percentage of total amines that are acetylated in polyethylenimine. In some embodiments, the PEI is branched and acetylated. In some embodiments, the branched PEI that is used as a reagent in the acetylation reaction has a molecular weight of about 25 kDa (Millipore Sigma, St. Louis, MO). In some embodiments, the percentage acetylation of PEI is from about 10% to about 60%. In some embodiments, the percentage acetylation of PEI is from about 20% to about 50%. In some embodiments, the percentage acetylation of PEI is from about 20% to about 25%. In some embodiments, the percentage acetylation of PEI is about 25%.

The term “N/P ratio” refers to the molar ratio of nitrogen (N) in the amine containing cationic biomaterial, such as polyethylenimine, to the total phosphate (P) in nucleic acid. In some embodiments, the non-viral polyplex particle has an N/P ratio of at least 7.5. In some embodiments, the non-viral polyplex particle has an N/P ratio of at least 15. In some embodiments, the non-viral polyplex particle has an N/P ratio of at least 30.

In some embodiments, the N/P ratio of the polyplex particles can be optimized to transfect specific cell types, such as immune cells. In some embodiments, polyplex particles have an N/P ratio of at least 15 for transfecting macrophages. In some embodiments, polyplex particles have an N/P ratio of at least 7.5 for transfecting T cells. In preferred embodiments, polyplex particles comprising an amine containing cationic biomaterial such as acetylated PEI and an anionic biomaterial such as PEMA have an N/P ratio of about 30. In preferred embodiments, the cells to be transfected are immune cells.

In some embodiments, the weight percent of the amine containing cationic biomaterial complexed with nucleic acid in the particle ranges from about 50% to about 99.9%. In some embodiments, the weight percent of the amine containing cationic biomaterial complexed with nucleic acid in the particle ranges from about 66% to about 90%. In some embodiments, the weight percent of the amine containing cationic biomaterial complexed with nucleic acid in the particle is about 50%, about 55%, about 60%, about 65%, about 70%, about 75%, about 80%, about 85%, about 90%, about 95%, or about 99%.

In some embodiments, the anionic biomaterial is selected from the group consisting of poly(glutamic acid), poly(aspartic acid), poly(acrylic acid), hyaluronic acid, poly(methyl vinyl ether-alt-maleic acid), poly(isobutylene-alt-maleic acid), poly(ethylene-alt-maleic acid), poly(ethylene-alt-maleic anhydride), and combinations thereof. In some embodiments, the anionic biomaterial is poly(ethylene-alt-maleic acid).

In some embodiments, the polyplex particle comprises acetylated branched PEI as the amine containing cationic biomaterial and poly(ethylene-alt-maleic anhydride) (PEMA) as the anionic biomaterial.

In some embodiments, the anionic biomaterial is present in the polyplex particle at a weight percent of about 0.1% to about 67%. In some embodiments, the anionic biomaterial is present in the polyplex particle at a weight percent of about 0.1% to about 50%. In some embodiments, the anionic biomaterial is present at a weight percent of about 1% to about 40%. In some embodiments, the anionic biomaterial is present at a weight percent of about 2% to about 35%. In some embodiments, the anionic biomaterial is present at a weight percent of about 5% to about 35%. In some embodiments, the anionic biomaterial is present at a weight percent of about 5% to about 25%. In some embodiments, the anionic biomaterial is present at a weight percent of about 10% to about 20%.

In some embodiments, the particle does not comprise a targeting moiety. In some embodiments, the non-viral polyplex particle has a surface zeta potential of at least about +10. In some embodiments, the non-viral polyplex particle has a zeta potential of between about +25 to about +30. In some embodiments, the non-viral polyplex particle has a zeta potential less than about +50.

In some embodiments, the non-viral polyplex particle further comprises a targeting moiety that enables delivery of the nucleic acids to a target cell, wherein the targeting moiety binds to the surface of the target cell, wherein the non-viral polyplex particle is internalized by the target cell by receptor mediated endocytosis.

In some embodiments, the targeting moiety is selected from the group consisting of a protein, a cell adhesion molecule, an antibody, a peptide, a sugar, a small molecule, and any combination thereof.

In some embodiments, the targeting moiety comprises a single chain antibody. In some embodiments, the targeting moiety comprises a single chain (scFv) variable fragment antibody.

In some embodiments, the polyplex particle comprises a targeting moiety that binds to a cell surface molecule or complex on an immune cell. In some embodiments, the targeting moiety binds to a cell surface molecule or complex selected from the group consisting of mannose receptor (CD206), folic acid receptor, Ly6G, Ly6C and CD3. The targeting moieties could be various types of targeting ligands such as mannose, folic acid, tuftsin peptide, anti-Thy1.1. In some embodiments the targeting moiety is an anti-CD3e F(ab)₂ molecule. In some embodiments, the targeting moiety is conjugated to the anionic biomaterial, thereby enabling presentation of the targeting moiety at the outer surface of the polyplex particle. In some embodiments, the targeting moiety is conjugated to poly(ethylene-alt-maleic anhydride).

In some embodiments, the polyplex particle comprises a targeting moiety that binds to a cell surface molecule or complex on a cancer cell.

The nucleic acid that can be delivered to cells in the polyplex particle in the compositions and methods of the invention is not particularly limiting. The terms “nucleic acid,” and “polynucleotide,” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties.

In some embodiments, the nucleic acid encodes a polypeptide. The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids. Such polypeptides can be wild-type or a variant, modified or chimeric polypeptide. A “variant polypeptide” can mean a modified polypeptide such that the modified polypeptide has an amino acid alteration compared to wild-type polypeptide.

The term “sequence” relates to a nucleotide sequence of any length, which can be DNA or RNA; can be linear, circular or branched and can be either single-stranded or double stranded.

In some embodiments, the nucleic acid is selected from the group consisting of DNA, messenger RNA, small interfering RNA (siRNA), short hairpin RNA (shRNA), antisense oligonucleotides (ODN) and combination thereof. In some embodiments, the nucleic acid is selected from the group consisting of an antisense oligonucleotide, cDNA, genomic DNA, guide RNA, plasmid DNA, vector DNA, mRNA, miRNA, piRNA, shRNA, lncRNA, siRNA, and combinations thereof. In some embodiments, the nucleic acid is not siRNA. In some embodiments, the DNA comprises plasmid DNA. As used herein, the term “plasmid DNA” refers to a small DNA molecule that is typically circular and is capable of replicating independently.

In some embodiments, the nucleic acid encodes a fragment of a polypeptide. In some embodiments, the nucleic acid comprises a template nucleic acid that can be useful, e.g., to modify the genome of the cell. In some embodiments, the nucleic acids encode one or more nucleases useful for modifying the genome of the cell.

In some embodiments, the nucleic acid encodes or modulates the expression of one or more proteins. Non-limiting examples include a blood clotting factor (e.g., Factor XIII, Factor IX, Factor X, Factor VIII, Factor VIIa, or protein C), apoE2, TPP1, argininosuccinate synthase, copper transporting ATPase 2, acid alpha-glucosidase, β-Glucocerebrosidase, α-galactosidase, C1 inhibitor serine protease inhibitor, CFTR (cystic fibrosis transmembrane regulator protein), an antibody, retinal pigment epithelium-specific 65 kDa protein (RPE65), erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor (e.g., insulin-like growth factors 1 and 2, platelet derived growth factor, epidermal growth factor, nerve growth factor, neurotrophic factor-3 and -4, brain-derived neurotrophic factor, glial derived growth factor, transforming growth factor .alpha. and .beta., etc.), a suicide gene product (e.g., herpes simplex virus thymidine kinase, cytosine deaminase, diphtheria toxin, cytochrome P450, deoxycytidine kinase, tumor necrosis factor, etc.), a drug resistance protein (e.g., that provides resistance to a drug used in cancer therapy), a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitopes, or hCDR1, insulin, glucokinase, guanylate cyclase 2D (LCA-GUCY2D), Rab escort protein 1 (Choroideremia), LCA 5 (LCA-Lebercilin), ornithine ketoacid aminotransferase (Gyrate Atrophy), Retinoschisin 1 (X-linked Retinoschisis), USH1C (Usher's Syndrome 1C), X-linked retinitis pigmentosa GTPase (XLRP), MERTK (AR forms of RP: retinitis pigmentosa), DFNB1 (Connexin 26 deafness), ACHM 2, 3 and 4 (Achromatopsia), PKD-1 or PKD-2 (Polycystic kidney disease), TPP1, CLN2, gene deficiencies causative of lysosomal storage diseases (e.g., sulfatases, N-acetylglucosamine-1-phosphate transferase, cathepsin A, GM2-AP, NPC1, VPC2, Sphingolipid activator proteins, etc.), one or more zinc finger nucleases for genome editing, and/or donor sequences used as repair templates for genome editing.

In some embodiments, the nucleic acid encodes or modulates the expression of one or more cytokines. In some embodiments, the nucleic acid reduces expression of an endogenous cytokine. In some embodiments, the cytokine is selected from the group consisting of transforming growth factor-beta (TGF-beta), interferons (e.g., interferon-alpha, interferon-beta, interferon-gamma), colony stimulating factors (e.g., granulocyte colony stimulating factor (GM-CSF)), thymic stromal lymphopoietin (TSLP), and the interleukins, e.g., interleukin-1, interleukin-2, interleukin-3, interleukin-4, interleukin-5, interleukin-6, interleukin-7, interleukin-8, interleukin-10, interleukin-12, interleukin-13, interleukin-15, interleukin-17, interleukin-18, interleukin-22, interleukin-23, and interleukin-35.

In some embodiments, the nucleic acid encodes a therapeutic antibody or an Fc fusion protein useful in the treatment of an inflammatory disease. Anti-inflammatory antibodies include adalimumab, alemtuzumab, atlizumab, canakinumab, certolizumab, certolizumab pegol, daclizumab, efalizumab, fontolizumab, golimumab, infliximab, mepolizumab, natalizumab, omalizumab, ruplizumab, ustekinumab, visilizumab, zanolimumab, vedolizumab, belimumab, otelixizumab, teplizumab, rituximab, ofatumumab, ocrelizumab, epratuzumab, eculizumab, and briakinumab. Exemplary useful Fc fusion proteins to treat inflammatory diseases include atacicept, abatacept, alefacept, etanercept, and rilonacept.

In some embodiments, the nucleic acid modulates expression of one or more polypeptide hormones. In some embodiments, the nucleic acid encodes the polypeptide hormone. In some embodiments, the nucleic acid reduces expression of an endogenous polypeptide hormone. In some embodiments, the polypeptide hormone is selected from the group consisting of amylin, anti-Mullerian hormone, calcitonin, cholecystokinin, corticotropin, endothelin, enkephalin, erythropoietin (EPO), follicle-stimulating hormone, gallanin, gastrin, ghrelin, glucagon, gonadotropin-releasing hormone, growth hormone-releasing hormone, hepcidin, human chorionic gonadotropin, human growth hormone (hGH), inhibin, insulin, insulin-like growth factor, leptin, luteinizing hormone, luteinizing hormone releasing hormone, melanocyte stimulating hormone, motilin, orexin, oxytocin, pancreatic polypeptide, parathyroid hormone, prolactin, secretin, somatostatin, thrombopoietin, thyroid-stimulating hormone, vasoactive intestinal peptide, and vasopressin.

In some embodiments, the nucleic acid (e.g., siRNA, miR, mRNA) is capable of modulating the expression and/or activity of one or more proteins (e.g., an enzyme, e.g., a kinase) associated with a cancer. In some embodiments, the nucleic acid can target a protein selected from the group consisting of: kinesin spindle protein (KSP), RRM2, keratin 6a (K6a), HER1, ErbB2, a vascular endothelial growth factor (VEGF) (e.g., VEGFR1, VEGFR3), a platelet-derived growth factor receptor (PDGFR) (e.g., PDGFR-α, PDGFR-β), epidermal growth factor receptor (EGFR), a fibroblast growth factor receptor (FGFR) (e.g., FGFR1, FGFR2, FGFR3, FGFR4), EphA2, EphA3, EphA4, HER2, HER3, HER4, INS-R, IGF-1R, IR-R, CSF1R, KIT, FLK-II, KDR/FLK-1, FLK-4, fit-1, c-Met, Ron, Sea, TRKA, TRKB, TRKC, FLT3, VEGFR/Flt2, Flt4, EphA1, EphB2, EphB4, Pim1, Pim2, Pim3, Tie2PKN3, PLK1, PLK2, PLK3, Src, Fyn, Lck, Fgr, Btk, Fak, SYK, FRK, JAK, Abl, Kit, KDR, CaM-kinase, phosphorylase kinase, MEKK, ERK, mitogen activated protein (MAP) kinase, phosphatidylinositol-3-kinase (PI3K), an AKT (e.g., Akt1, Akt2, Akt3), TGF-$R, KRAS, BRAF, a cyclin-dependent kinase (e.g., CDK1, CDK2, CDK4, CDK5, CDK6, CDK7, and CDK9), GSK3, a CDC-like kinase (CLK) (e.g., CLK1, CLK4), an Aurora kinase (e.g., Aurora A, Aurora B, and Aurora C), a mitogen-activated protein kinase (MEK) (e.g., MEK1, MEK2), mTOR, protein kinase A (PKA), protein kinase C (PKC), protein kinase G (PKG), and PHB1.

In some embodiments, the nucleic acid encodes an antibody molecule. In some embodiments, the nucleic acid encodes a therapeutic antibody molecule capable of treating cancer. In some embodiments, the nucleic acid sequence encodes variable domain sequences of an antibody selected from abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, and zalutumumab.

In some embodiments, the nucleic acid encodes a chimeric antigen receptor (CAR). In some embodiments, the CAR may include an antigen binding domain, a transmembrane domain, a costimulatory signaling region, and a CD3 zeta signaling domain. In some embodiments, the antigen binding domain may bind to an antigen of a non-essential organ.

The CAR can be expressed in a target cell selected from the group consisting of a T cell, a natural killer (NK) cell, NK-92 cell, a cytotoxic T lymphocyte (CTL), and a regulatory T cell.

CARs are molecules generally including an extracellular and intracellular domain. The extracellular domain includes a target-specific binding element. The intracellular domain (e.g., cytoplasmic domain) includes a costimulatory signaling region and a zeta chain portion. The costimulatory signaling region refers to a portion of the CAR including the intracellular domain of a costimulatory molecule. Costimulatory molecules are cell surface molecules other than antigens receptors or their ligands that are required for an efficient response of lymphocytes to antigen. Between the extracellular domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain of the polypeptide chain. A spacer domain may include up to 300 amino acids, preferably 10 to 100 amino acids, and most preferably 25 to 50 amino acids.

In some embodiments, the target-specific binding element of the CAR in the present disclosure may recognize a tumor antigen. Tumor antigens are proteins that are produced by tumor cells that elicit an immune response, particularly T-cell mediated immune responses. Tumor antigens are well known in the art and include, for example, a glioma-associated antigen, carcinoembryonic antigen (CEA), β-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxylesterase, mut hsp70-2, M-CSF, prostase, prostate-specific antigen (PSA), PAP, NY-ESO-1, LAGE-la, p53, prostein, PSMA, Her2/neu, survivin and telomerase, prostate carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor .alpha. 2, IL-11 receptor .alpha., MUC1, MUC16, CA9, GD2, GD3, HMW-MAA, CD171, Lewis Y, G250/CAIX, HLA-AI MAGE A1, HLA-A2 NY-ESO-1, PSC1, folate receptor-.alpha., CD44v7/8, 8H9, NCAM, VEGF receptors, 514, Fetal AchR, NKG2D ligands, CD44v6, TEM1, TEM8, or viral-associated antigens expressed by the tumor.

In some embodiments, the binding element of the CAR may include any antigen binding moiety that when bound to its cognate antigen, affects a tumor cell such that the tumor cell fails to grow, or is promoted to die or diminish.

In some embodiments, the nucleic acid encodes or is an RNA interfering agent. As used herein, an “RNA interfering agent” is defined as any agent that interferes with or inhibits expression of a target gene, e.g., by RNA interference (RNAi). Such RNA interfering agents include, but are not limited to, antisense molecules, ribozymes, small inhibitory nucleic acid sequences, for example but not limited to guide RNAs, small interfering RNA (siRNA), short hairpin RNA or small hairpin RNA (shRNA), microRNA (miRNA), post-transcriptional gene silencing RNA (ptgsRNA), short interfering oligonucleotides, antisense oligonucleotides, aptamers, CRISPR RNAs, nucleic acid molecules including RNA molecules which are homologous to the target gene, or a fragment thereof, and any molecule which interferes with or inhibits expression of a target gene by RNA interference (RNAi).

Non-limiting examples of target genes where it is desirable to inhibit expression include huntingtin (HTT) gene, a gene associated with dentatorubropallidolusyan atrophy (e.g., atrophin 1, ATN1); androgen receptor on the X chromosome in spinobulbar muscular atrophy, human Ataxin-1, -2, -3, and -7, Cav2.1 P/Q voltage-dependent calcium channel is encoded by the (CACNA1A), TATA-binding protein, Ataxin 8 opposite strand, also known as ATXN8OS, Serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; Myotonin-protein kinase (MT-PK) in myotonic dystrophy; Frataxin in Friedreich's ataxia; a mutant of superoxide dismutase 1 (SODi) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercoloesterolemia; HIV Tat, human immunodeficiency virus transactivator of transcription gene, in HIV infection; HIV TAR, HIV TAR, human immunodeficiency virus transactivator response element gene, in HIV infection; C—C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DAN damage-inducible transcript 4 protein, in diabetic macular oedma (DME) or age-related macular degeneration; vascular endothelial growth factor receptor I (VEGFR1) in age-related macular degeneration or choroidal neovascularization, caspase 2 in non-arteritic ischaemic optic neuropathy; Keratin 6A N17K mutant protein in pachyonychia congenital; influenza A virus genome/gene sequences in influenza infection; severe acute respiratory syndrome (SARS) coronavirus genome/gene sequences in SARS infection (e.g., spike protein in SARS-CoV-2); respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection, coxsackievirus B3 genome/gene sequences in coxsackievirus B3 infection; silencing of a pathogenic allele of a gene (allele-specific silencing) like torsin A (TOR1A) in primary dystonia, pan-class I and HLA-allele specific in transplant; mutant rhodopsin gene (RHO) in autosomal dominantly inherited retinitis pigmentosa (adRP); or the inhibitory nucleic acid binds to a transcript of any of the foregoing genes or sequences.

### Pharmaceutical Compositions

When employed as pharmaceuticals, the polyplex particles of the invention can be administered in the form of pharmaceutical compositions. Thus the present disclosure provides a composition comprising a polyplex particle, or a pharmaceutically acceptable salt thereof, or any of the embodiments thereof, and at least one pharmaceutically acceptable carrier. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject and which does not destroy the pharmacological activity of the polyplex particle and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

This invention also includes pharmaceutical compositions which contain, as the active ingredient, the polyplex particle of the invention or a pharmaceutically acceptable salt thereof, in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active particle, soft and hard gelatin capsules, suppositories, sterile injectable solutions and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the subject by employing procedures known in the art.

In some embodiments, the pharmaceutical composition comprises silicified microcrystalline cellulose (SMCC) and at least one polyplex particle described herein, or a pharmaceutically acceptable salt thereof. In some embodiments, the silicified microcrystalline cellulose comprises about 98% microcrystalline cellulose and about 2% silicon dioxide w/w.

In some embodiments, the compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S.

Food and Drug Administration. For example, suitable formulations may be sterile and/or substantially isotonic and/or in full compliance with all Good Manufacturing Practice regulations of the U.S. Food and Drug Administration.

In some embodiments, the active polyplex particle may be effective over a wide dosage range and is generally administered in a therapeutically effective amount. It will be understood, however, that the amount of the particle actually administered will usually be determined by a physician, according to the relevant circumstances, including the condition to be treated, the chosen route of administration, the actual particle administered, the age, weight, and response of the individual subject, the severity of the subject's symptoms and the like.

In some embodiments, the therapeutic dosage of a polyplex particle of the present invention can vary according to, e.g., the particular use for which the treatment is made, the manner of administration of the particle, the health and condition of the subject, and the judgment of the prescribing physician. The proportion or concentration of a particle of the invention in a pharmaceutical composition can vary depending upon a number of factors including dosage, chemical characteristics (e.g., hydrophobicity), and the route of administration. For example, the particles of the invention can be provided in an aqueous physiological buffer solution containing about 0.1 to about 10% w/v of the particle for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular subject, the relative biological efficacy of the particle selected, formulation of the excipient, and its route of administration. Effective doses can be extrapolated from dose-response curves derived from in vitro or animal model test systems.

In some embodiments, the liquid forms in which the polyplex particles and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases.

Nebulized solutions may be breathed directly from the nebulizing device or the nebulizing device can be attached to a face mask, tert, or intermittent positive pressure breathing machine. Solution, suspension, or powder compositions can be administered orally or nasally from devices which deliver the formulation in an appropriate manner.

In some embodiments, topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the polyplex particles of the invention. The topical formulations can be suitably packaged in tubes of, e.g., 100 g which are optionally associated with instructions for the treatment of the select indication, e.g., psoriasis or other skin condition.

In some embodiments, the amount of polyplex particle or composition administered to a subject will vary depending upon what is being administered, the purpose of the administration, such as prophylaxis or therapy, the state of the subject, the manner of administration and the like. In therapeutic applications, compositions can be administered to a subject already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the subject and the like.

In some embodiments, the compositions administered to a subject can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the particle preparations typically will be between 3 and 11, more preferably from 5 to 9 and most preferably from 7 to 8. It will be understood that use of certain of the foregoing excipients, carriers or stabilizers will result in the formation of pharmaceutical salts.

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

The present invention is further illustrated by the following Examples. These Examples are provided to aid in the understanding of the invention and are not to be construed as a limitation thereof.

### EXAMPLES

### Example 1: Serum-Independent Non-Viral Gene Delivery to Innate and Adaptive Immune Cells Using Immunoplexes

### Experimental Section:

Materials: Acetic anhydride, triethylamine, and branched polyethylenimine (PEI; MW 25 kDa) was purchased from Millipore Sigma (St. Louis, MO). Poly(ethylene-alt-maleic anhydride) (PEMA) ZeMAC™ E60 (PEMA) was received as a gift from Vertellus™ (Indianapolis, IN) was purchased from Polyscience, Inc. (Warrington, PA), NHS-Rhodamine (NHS-RHO) was purchased from Fisher Scientific (Waltham, MA). Maxiprep Endotoxin Free Kit was purchased from Qiagen (Germantown, MD). Unless otherwise noted, any additional reagents were purchased from Millipore Sigma.

Cell culture: RAW 264.7 cells were cultured in Dulbecco's Modified Eagle Medium (DMEM) (Millipore Sigma; St. Louis, MO) supplemented with 10% heat-inactivated fetal bovine serum (FBS) (VWR; Radnor, PA) and 1% penicillin/streptomycin (P/S) (Invitrogen Corporation; Carlsbad, CA). DC2.4 cells were cultured in in RPMI 1640 (Millipore Sigma; St. Louis, MO) supplemented with 10% FBS, 1% P/S, and 50 μM β-mercaptoethanol. Jurkat cells were cultured in RPMI 1640 supplemented with 10% FBS and 1% P/S. Both cell lines were incubated at 37° C. and 5% CO₂.

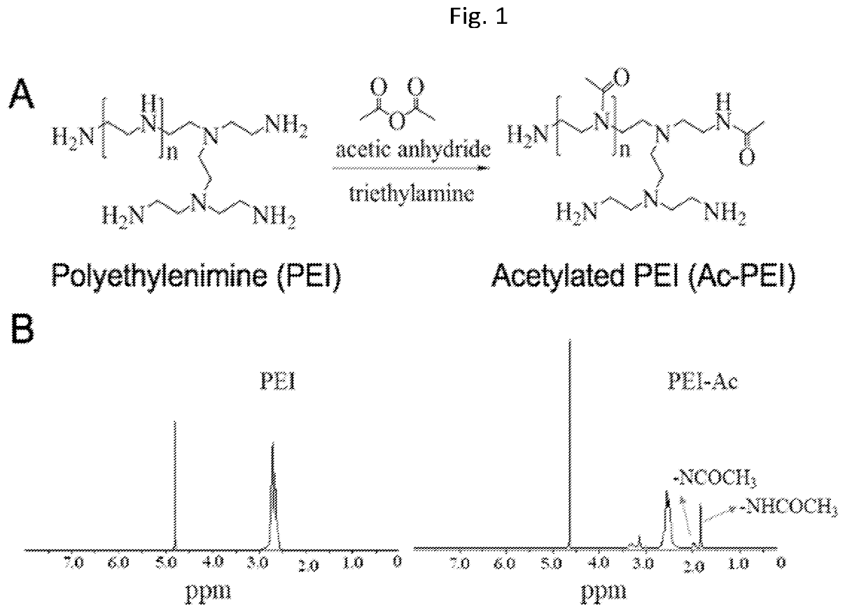

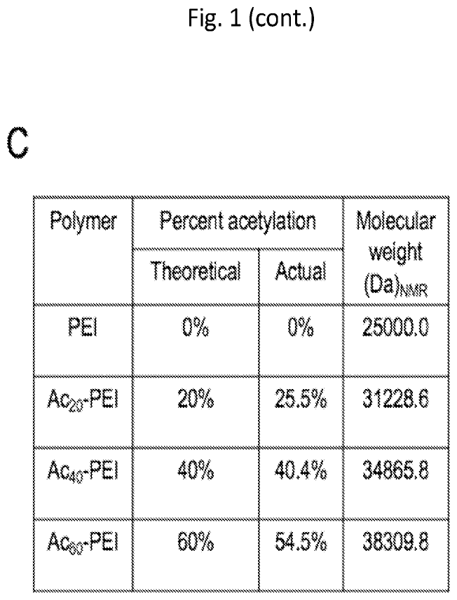

Synthesis of Ac-PEI and rhodamine-conjugated PEI (Ac-PEI-RHO): Ac-PEI with varying degrees of acetylation was synthesized using a previously reported method (Forrest et al., Pharm Res 2004, 21, 365-71.). Briefly, 500 mg of PEI (0.02 mmol, 25000 g/mol) was added to 20 mL scintillation vials and dissolved in 10 mL methanol. Triethylamine (5 molar equivalents to acetic anhydride) was then added followed by acetic anhydride (FIG. 1C) to achieve theoretical degrees of acetylation of 20%, 40%, and 60% of the total amines (Ac₂₀-PEI, Ac₄₀-PEI, and Ac₆₀-PEI respectively). The reaction was allowed to stir overnight at room temperature and the modified PEI was purified by dialysis against water using a 12-14 kDa MWCO membrane and recovered by lyophilization. ¹H-NMR was used to determine the actual percent acetylation of PEI as previously reported (Forrest et al., Pharm Res 2004, 21, 365-71.). Ac-PEI polymer was dissolved in 800 μL of D₂O and the ¹H-NMR spectra was acquired using a 400 MHz Varian spectrometer. Fluorescently-labeled Ac-PEI was also synthesized by conjugating NHS-Rhodamine (RHO) with Ac-PEI. Briefly, 10 mg Ac₂₀-PEI (3.51×10−4 mmol) was dissolved in methanol and then 1 mg of NHS-RHO (1.89×10⁻³ mmol) and 50 μL of triethylamine was added dropwise and stirred for 4 h. After the reaction, the solution was dialyzed against water with a 12-14 kDa MWCO membrane and lyophilized for further use.

GFP Plasmid preparation: DH5u E. coli competent cells (Invitrogen Corporation; Carlsbad, CA) were transformed with eGFP (GFP) pDNA (courtesy of National Center for Toxicological Research, FDA, Jefferson, AR), which encoded for kanamycin resistance. Transformed cells were expanded in an overnight liquid LB culture at 37° C. under vigorous shaking, lysed, and purified using a Qiagen Maxiprep Endotoxin Free Kit. The concentration of pDNA was verified using a SpectraMax iD3 microplate reader and a SpectraDrop micro-volume microplate (Molecular Diagnostics; San Jose, CA) by measuring absorbance at 260 nm and 280 nm and agarose gel electrophoresis. pDNA was stored at −20° C. until further use.

Stability of Ac₂₀-PEI, Ac₄₀-PEI, and Ac₆₀-PEI polyplexes: The binding capacity of polymers to DNA was analyzed by DNA gel electrophoresis. Fresh polyplex solutions were prepared immediately prior to each experiment. Polyplexes were prepared using Ac₂₀-PEI, Ac₄₀-PEI, and Ac₆₀-PEI, at different N/P ratios ranging from 0 to 20. First, plasmid DNA was diluted in HEPES buffer (20 mM, pH 7) at a concentration 500 ng/mL. Fresh Ac-PEI was prepared at a concentration of 1 mg/mL in water. Next, the appropriate amount of Ac-PEI and plasmid DNA were mixed together in a microcentrifuge tube and incubated for 10 minutes at room temperature. 6× Loading dye (Invitrogen Corporation; Carlsbad, CA) was added to the polyplexes prior to running on a 1% agarose gel, followed by staining with ethidium bromide. pDNA was visualized with an Invitrogen™ E-Gel™ Imager System (Fisher Scientific; Waltham, MA).

Preparation of IPs: All IPs evaluated used Ac₂₀-PEI polymers and were complexed with pDNA at N/P ratios of 7.5, 15, and 30. The formed polyplexes are referred to as Ac-PEI (N/P ratio). Polyplexes were enveloped by adding PEMA (1 mg/mL) in HEPES at various weight ratios (10 wt %, 30 wt %, and 50 wt %) with respect to Ac-PEI followed by incubation at room temperature for 10-20 minutes to form IPs. IPs are denoted as IP_{(N/P ratio)}−(PEMA wt %). For example, IP₃₀-10 describes an IP prepared at N/P 30 and 10 wt % PEMA.

Hydrodynamic size and zeta potential measurement: Fresh polyplex and IPs solutions were prepared with 2 pg of GFP plasmid in HEPES buffer. 1 mL of each sample was added into a disposable cuvette and the size was measured using a Zetasizer Nano ZS (Malvern Instruments Inc.). 15 runs were performed in triplicate for each sample. Subsequently, samples were transferred to a folded capillary cell (Malvern) and zeta-potential measurements were performed in triplicate for each sample using the Zetasizer Nano ZS (Malvern).

Stability of IPs in presence of PEMA and serum: The stability of IPs is critical to ensure pDNA can be efficiently delivered to immune cells of interest in vivo without destabilization. The stability of IP₃₀-10, 30, 50 was assessed by DNA gel electrophoresis as described above in buffer or after incubating with 55% fetal bovine serum (FBS) in PBS, a physiologically-relevant serum concentration, for 30-60 minutes (Salvati et al., Nature Nanotechnology 2013, 8, 137-143).

In vitro transfection of RAW 264.7 macrophages, DC2.4 dendritic cells, and human Jurkat T cells: Polyplexes and corresponding IPs were prepared at N/P 30 containing 2 pg of pDNA encoding GFP (Ac-PEI30). RAW 264.7, DC2.4, or Jurkat cells were treated for 4 h in serum-containing or serum-free media followed by washing to remove excess complex prior to overnight incubation in complete media. RAW 264.7 and DC2.4 cells are adherent and excess complex was easily removed by washing with PBS and replaced with complete DMEM or RPMI1640, respectively. In contrast, Jurkat cells are suspension cells and were washed by first transferring to a microcentrifuge tube followed by centrifugation at 500×g for 5 min to separate from polyplexes and IPs. The supernatant was removed, and the cell pellet was dispersed in a fresh complete media. Rhodamine and GFP expression was visualized using a Revolve fluorescence microscope (ECHO, San Diego, CA) at 24 h post-transfection. The cellular interactions and uptake were determined using polyplex and IPs prepared with Ac₂₀-PEI-RHO.

Flow cytometry: RAW 264.7, DC2.4, or Jurkat cells were cultured in a 24-well plate at a density 2×10⁵ cells/well for 24 h. Next, cells were transfected with different formulations of IPs in both serum-free and serum-containing media for 4 h. Subsequently, cells were washed and incubated for another 24 h in fresh serum-containing media. After 24 h, cells were collected using a cell scraper followed by centrifugation at 500×g for 5 minutes (RAW 264.7 and DC2.4 cells are adherent) or by centrifugation at 500×g for 5 minutes (Jurkat cells are in suspension) and resuspended in fresh flow cytometry buffer. For analysis of live cells only, 4′,6-diamidino-2-phenylindole dilactate (DAPI) was used as an exclusion dye to determine cell viability. Data was collected using an LSR II (Becton-Dickinson, San Jose, CA) flow cytometer and analyzed by FCS Express 7 (De Novo Software, Pasadena, CA) and transfection efficiency was measured as the percentage of live cells which were GFP⁺ compared to un-transfected controls.

In vitro persistence of GFP expression: Raw 264.7 cells were grown in a 24-well plate at a density 2×10⁵ cells/well for 24 h in complete DMEM. After that, cells were transfected with Ac-PEI30, IP₃₀-10, IP₃₀-30 and IP₃₀-50 for 4 h in serum-containing medium. The cells were then washed and incubated with fresh DMEM for 4 h, 24 h, 48 h, and 72 h. The percentage of live cells which were GFP⁺ compared to controls was determined using flow cytometry.

MTS assay for assessing cytotoxicity: The cytotoxicity of IPs was evaluated using an MTS assay (Abcam; Cambridge, MA). RAW 264.7 and Jurkat cells were cultured in a 24-well plate at a density 2×10⁵ cells/well overnight prior to treatment with PEI variants [Ac-PEI7.5, Ac-PEI15, Ac-PEI30, PEI30] or IPs [IP₃₀-10, IP₃₀-30 and IP₃₀-50] for 4 h in both serum-containing and serum-free media. Following the incubation, cells were washed and further incubated with fresh media for 24 h. Next, 50 μL of MTS solution was added in each well and incubated for another 3 h. The optical density (O.D.) of the solution was measured using a SpectraMax iD3 microplate reader at 570 nm. The percentage of cell viability was measured as the ratio of O.D. at 570 nm to no treatment control. Each treatment was replicated for a total of three times.

### Results and Discussion

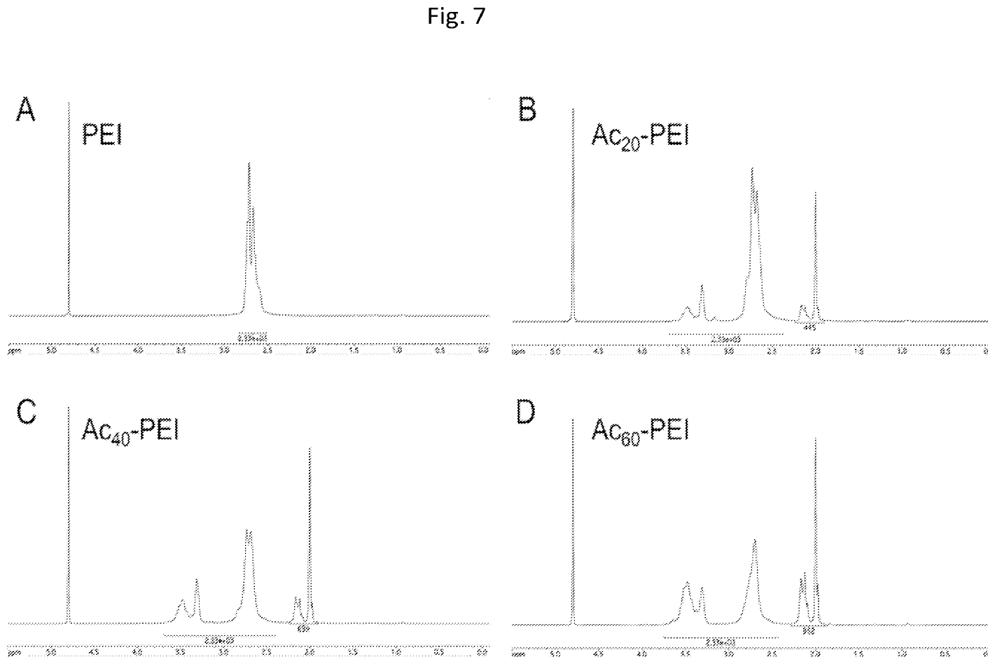

Synthesis and characterization of Ac-PEI polyplexes: Ac-PEI was synthesized by reacting 25 kDa branched PEI with various amounts of acetic anhydride to yield three variations of Ac-PEI (FIG. 1A). The primary and secondary amine groups of PEI react with acetic anhydride to form secondary and tertiary amides, respectively. ¹H-NMR was used to determine the total percent of acetylation, where the peak at 1.7-1.75 ppm signifies the acetylation of primary amine groups and the peak at 1.8-1.85 ppm depicts the acetylation of secondary amine groups of PEI (FIG. 1B and FIG. 7) (Forres et al., Pharm Res 2004, 21, 365-71). The actual percentage of acetylation for Ac₂₀-PEI, Ac₄₀-PEI, and Ac₆₀-PEI was measured to be 25.5%, 40.4% and 54.5%, respectively (FIG. 1C).

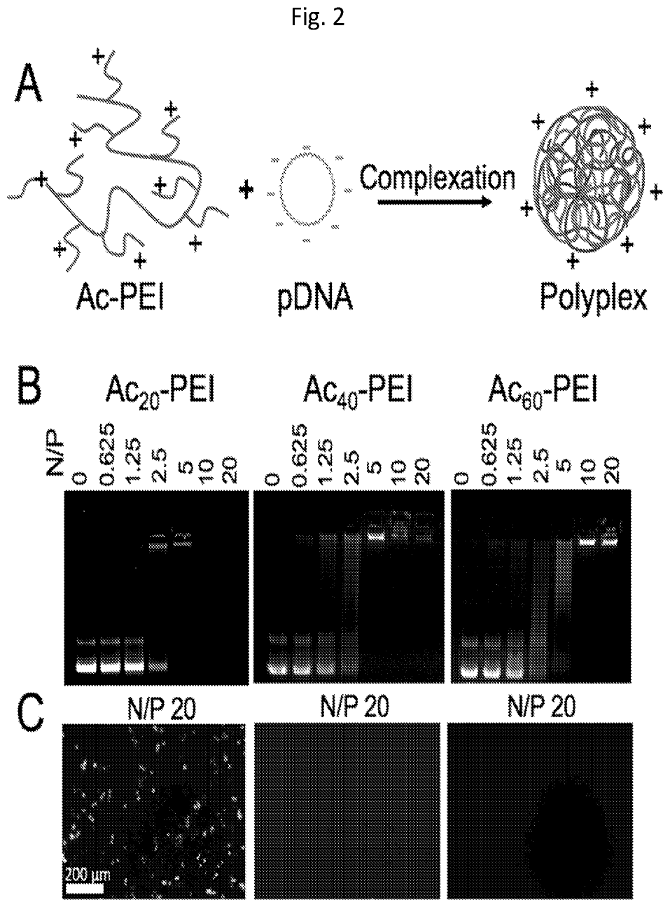

Polyplexes were formed at various N/P ratios to assess the effect of percentage of acetylation of Ac-PEI to form stable polyplexes with pDNA using DNA gel electrophoresis (FIGS. 2A and B). Ac₂₀-PEI formed more stable complexes than Ac₄₀-PEI and Ac₆₀-PEI, which was consistent with previous reports that polyplexes prepared at higher percentages of acetylation were less able to condense pDNA (Forres et al., Pharm Res 2004, 21, 365-71; Gabrielson et al., Biomacromolecules 2006, 7, 2427-2435). Further, the degree of acetylation correlated with ability of Ac₂₀-PEI, Ac₄₀-PEI, and Ac₆₀-PEI to transfect RAW 264.7 cells in vitro (FIG. 2C). Transfection is largely dependent on two factors, endosomal buffering capacity of the polyplexes, which aids in endosomal escape, and dissociation/unpackaging of the polyplexes intracellularly to release pDNA inside the cells (Forres et al., Pharm Res 2004, 21, 365-71; Gabrielson et al., Biomacromolecules 2006, 7, 2427-2435). Acetylation of PEI can enhance the dissociation of the polyplexes inside cells but also decreases the endosomal escape efficiency (Gabrielson et al., Biomacromolecules 2006, 7, 2427-2435). A lower percentage of acetylation with Ac₂₀-PEI enables retention of strong endosomal buffering capacity, whereas the polyplexes prepared with high levels of acetylation are not as effective (Gabrielson et al., Biomacromolecules 2006, 7, 2427-2435). Furthermore, acetylation of PEI also enhances the lipophilicity of PEI which can lead to enhancement of transfection (Thomas et al., Proc Natl Acad Sci USA 2002, 99, 14640-5). Due to the higher stability and transfection efficiency, Ac₂₀-PEI (referred to as Ac-PEI) was used in all subsequent experiments.

Overcoming serum-induced reductions in transfection efficiency in RAW 264.7 and Jurkat cells through modulation of the N/P ratio: Immune cells, especially suspension cells, are notoriously difficulty to transfect and commercially available transfection reagents have only shown limited success (Olden et al., J Control Release 2018, 282, 140-147; Zhao et al., J Control Release 2012, 159, 104-10). Numerous barriers must be overcome to enable effective DNA delivery to cells (Pearson et al., MRS Bulletin 2014, 39, 227-237). Accordingly, a balance must be achieved between DNA protection, DNA release, and toxicity to efficiently induce gene expression (Grigsby et al., J R Soc Interface 2010, 7 Suppl 1, S67-82). Polyplexes prepared at high N/P ratios exhibit improved serum stability yet suffer from a reduced ability to release DNA intracellularly, increased non-specific cellular interactions, and high toxicity.

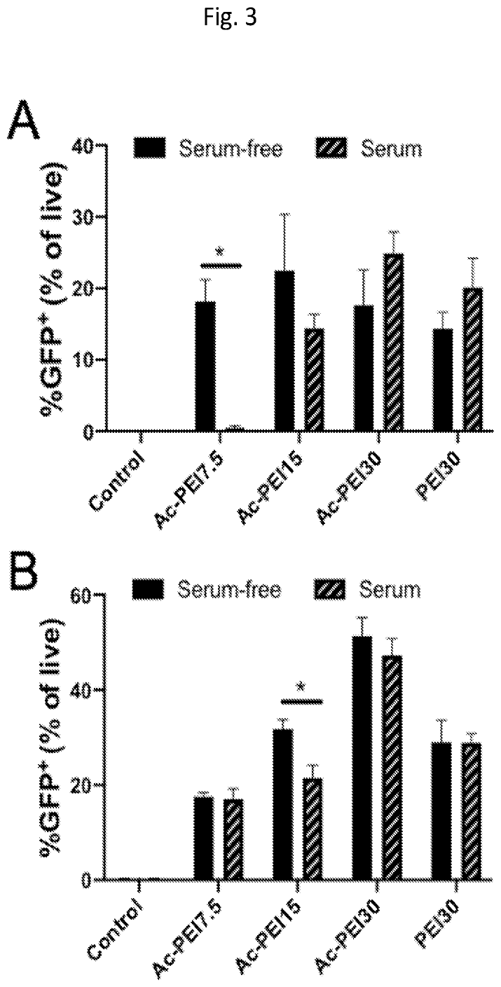

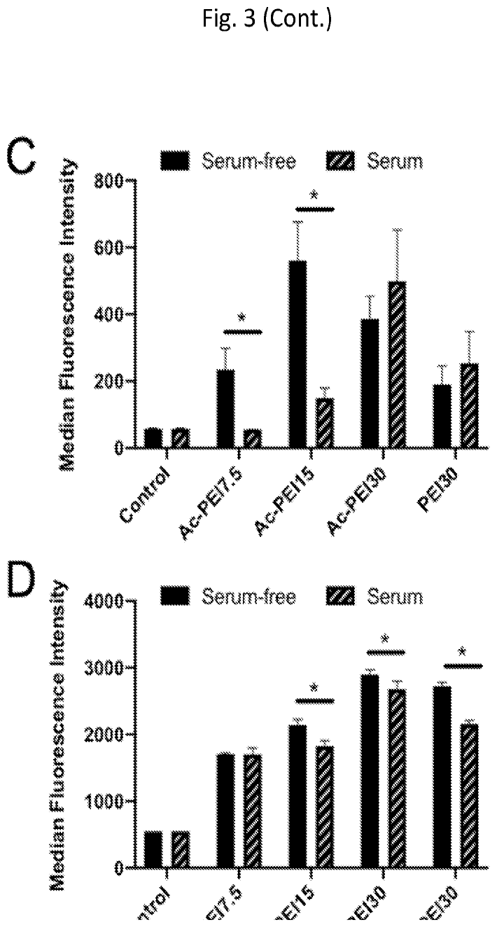

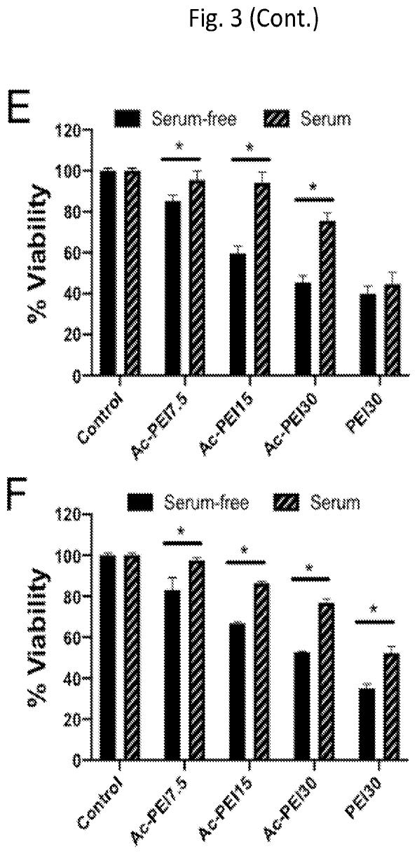

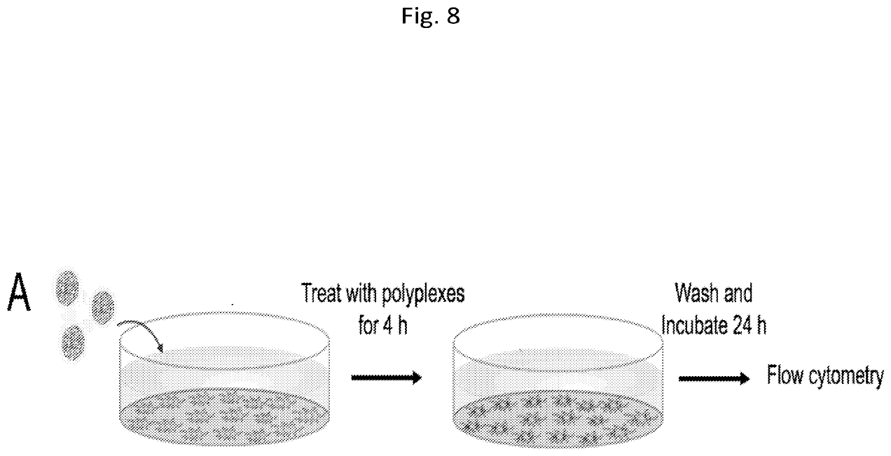

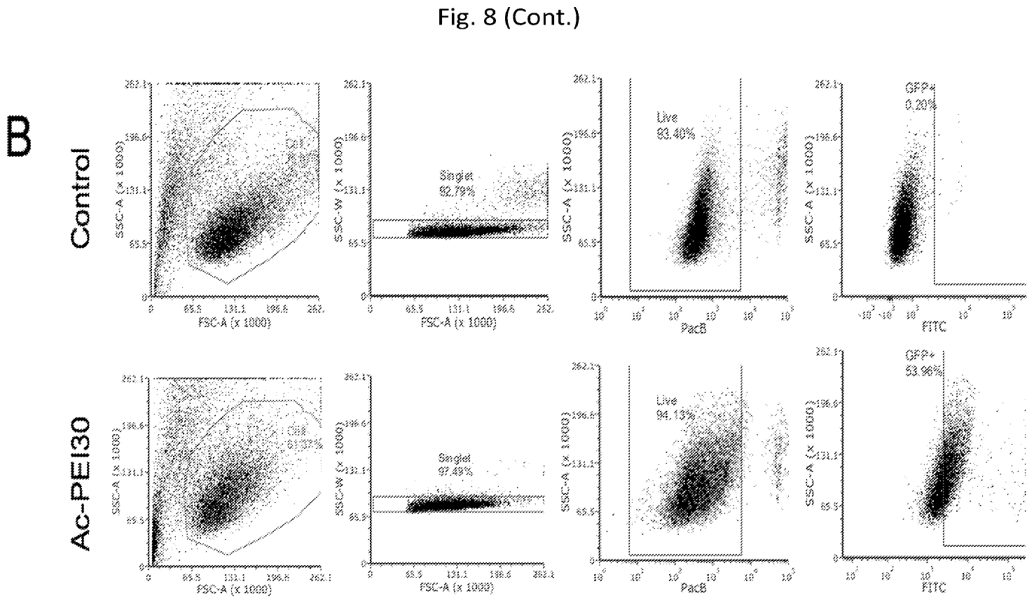

We assessed the impact of N/P ratio of Ac₂₀-PEI (Ac-PEI7.5, Ac-PEI15, Ac-PEI30) on the serum-dependent transfection of RAW 264.7 (adherent) and Jurkat (suspension) cells. The experimental setup as well as representative flow cytometry data are shown in FIG. 8. The impact of serum on transfection efficiency was more prominent in RAW 264.7 cells at low N/P ratios (FIG. 3A), whereas higher N/P ratios showed enhanced transfection efficiencies in and serum-free were less apparent for Jurkat cells, where increasing the N/P ratio from Ac-PEI7.5 to Ac-PEI30 resulted in enhanced transfection (FIG. 3B). Higher N/P ratios in the presence of serum are likely necessary due to the ability of free polymer to interact with serum proteins and assist with intracellular trafficking (Cai et al., J Control Release 2016, 238, 71-79; Boeckle et al., J Gene Med 2004, 6, 1102-11). Interestingly, the % GFP⁺ cells was similar for Ac-PEI30 and PEI30 in RAW 264.7 cells in serum-containing medium, the MFI for Ac-PEI30 was 96% higher than non-acetylated PEI30 controls (FIG. 3C). An opposite trend was observed for Jurkat cells where the % GFP⁺ was significantly higher for Ac-PEI30 versus PEI30, but the MFI of Ac-PEI30 was only increased by 25% (FIG. 3D). Critically, not only did Ac-PEI30 show significantly improved GFP expression compared to PEI30 overall, the improvement in cell viability of Ac-PEI30 compared to PEI30 was dramatic where a 70% increase in viability was noted for RAW 264.7 cells and a 47% increase was found for Jurkat cells (FIGS. 3E and F). Our results are in contrast to a previous study that found unmodified PEI could not increase the transfection efficiency in Jurkat cells likely because of its inability to unpackage the genetic payload at such high N/P ratios (Olden et al., J Control Release 2018, 282, 140-147). The improved GFP expression and overall cell viability of Ac-PEI30 in serum-containing medium (greater than 75%) compared to other polyplexes examined, provided our rationale to utilize Ac-PEI30 in our subsequent experiments.

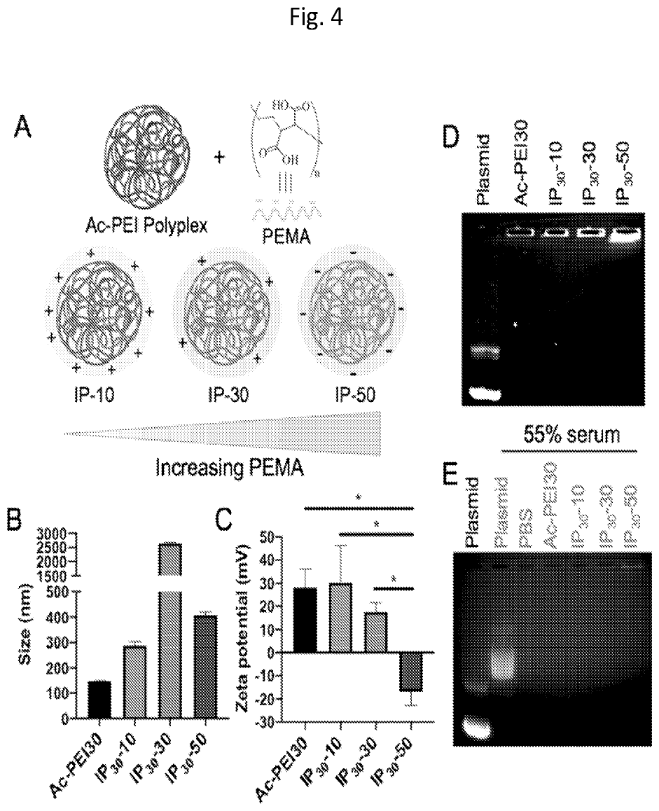

Enveloping polyplexes with PEMA (immunoplex (IP) formation) and physicochemical characterization: Polyplexes prepared at higher N/P ratios using Ac-PEI30 provided higher transfection efficiencies an improved toxicity profiles in serum-containing medium compared to PEI30 (FIG. 3). We hypothesized that by enveloping Ac-PEI30 polyplexes with the anionic polyelectrolyte PEMA, that we could further improve the toxicity profile at high N/P ratios and enhance the levels of gene expression by engineering improved mechanisms of endosomal escape into Ac-PEI30 through incorporation of a pH-dependent endosomal destabilization mechanism (Evans et al., Nat Commun 2019, 10, 5012). The polyplexes were enveloped with PEMA by first preparing Ac-PEI30 polyplexes at N/P 30 followed by the addition of PEMA at various weight ratios [10 wt % (IP₃₀-10), 30 wt % (IP₃₀-30), and 50 wt % (IP₃₀-50)] relative to Ac-PEI30 (FIG. 4A). The hydrodynamic diameter of Ac-PEI30 polyplexes was approximately 150 nm and +28 mV zeta potential (FIGS. 4B and C). As increased amounts of PEMA were incorporated, the surface charge of the IPs was decreased and IP₃₀-50 was −17 mV. The size increased with increasing percentage of PEMA. As the surface charge of IPs reached close to neutral (as in IP₃₀-30), aggregation was observed due to the loss of electrostatic repulsion between polyplexes. Further increases in PEMA such as IP₃₀-50 recovered the size distribution. A similar trend with size and zeta potential was observed for IPs prepared at N/P 7.5 and 15 (Table 1).

TABLE 1
Size and Zeta potentials of various IP
formulations prepared in this study.

| Immunoplexes | Size (nm), | Zeta Potential |
|---|---|---|
| (IP) | PDI | (mV) |
| Ac-PEI7.5 | 140.2, 0.123 | 15.6 |
| IP_{7.5}-10 | 146.5, 0.241 | 14.2 |
| IP_{7.5}-30 | 942.1, 0.328 | 5.8 |
| IP_{7.5}-50 | 242.5, 0.156 | −10.8 |
| Ac-PEI15 | 142.8, 0.118 | 20.4 |
| IP₁₅-10 | 145.9, 0.112 | 18.2 |
| IP₁₅-30 | 840.1, 0.420 | 10.2 |
| IP₁₅-50 | 348.3, 0.131 | −22.4 |

Stability of IPs in the presence of PEMA and physiologically-relevant serum concentrations: Engineering polyplexes with high stability is critical to ensure their genetic cargo is delivered appropriately to cell types of interest, particularly in vivo. The enveloping of cationic polyplexes with anionic polyelectrolyte coatings has the potential to reduce toxicity and minimize non-specific cellular interactions. However, enveloping could induce destabilization of the polyplexes through competition with negatively-charged DNA resulting in its subsequent release. To determine the effect of PEMA enveloping on IP stability, agarose gel electrophoresis was performed. FIG. 4D shows that at up to 50 wt % of PEMA, IPs maintained stable complexes, however IPs became unstable as the PEMA concentration was further increased (data not shown).

The effect of serum on IP stability was also confirmed by first formulating IPs followed by incubation in PBS containing 55% FBS, a physiologically-relevant serum concentration (Salvati et al., Nature Nanotechnology 2013, 8, 137-143; Gwak et al., Sci Rep 2017, 7, 11247). This experiment was performed to address two potential effects that may lead to reduced performance of IPs in vivo: 1) the formation of a biomolecule corona on the surface of polyplexes may induce IP destabilization through competition with negatively-charged DNA through the adsorption of serum proteins to the surface of the particles²⁷ and 2) FBS/serum contains nucleases that can degrade pDNA if not sufficiently protected by the polyplex (Pearson et al., Front Chem 2014, 2, doi: 10.3389/fchem.2014.00108; Gwak et al., Sci Rep 2017, 7, 11247.). FIG. 4E shows the stability of IPs was not affected by serum as indicated by a lack of DNA bands detected in the wells containing IPs. This result correlated with previous studies that have shown incubation of PEI-based polyplexes in high concentrations of FBS (greater than 50%) can maintain the stability of plasmid against degradation for at least 3 days (Gwak et al., Sci Rep 2017, 7, 11247.). Interestingly, the control well containing a mixture of serum and pDNA was able to partially condense the DNA through interactions between the negatively-charged DNA and positively-charged species present in the serum. These results demonstrated that the formulated polyplexes display beneficial stability during formulation and under in vivo-relevant conditions, which may allow for their successful in vivo application.

Controlled transfection of immune cells using IPs in serum-containing media: We hypothesized that PEMA enveloping of Ac-PEI/pDNA polyplexes would increase transfection efficiency of polyplexes prepared at high N/P ratios through improved unpackaging of DNA cargo and enhanced endosomal escape, while improving cell viability due to shielding of positive charges caused by the polyplexes. Enveloping of polyplexes with anionic polymers also has the potential to minimize the formation of non-specific interactions and enable cell-specific targeting to be engineered into the platform, thus improving its potential for in vivo applications. The transfection efficiency of rhodamine (RHO)-labeled IPs with variable coating with PEMA was assessed in vitro using RAW 264.7 and Jurkat cells. The uptake and transfection of IPs using RAW 264.7 and Jurkat cells in serum-containing medium is shown in Figures S3A and B, respectively. The uptake of IPs and the GFP protein expression decreased as a function of PEMA percentage in both cell types suggesting that PEMA enveloping reduced non-specific cellular interactions. Colocalization of RHO-labeled IPs and GFP signals were strongly visualized for RAW 264.7 cells and to a lesser extent for Jurkat cells due to difficulties associated with live cell imaging of suspension cells (Bäckström et al., bioRxiv 2020, 2020.01.05.895201; Tsang et al., Biotechniques 2017, 63, 230-233.).

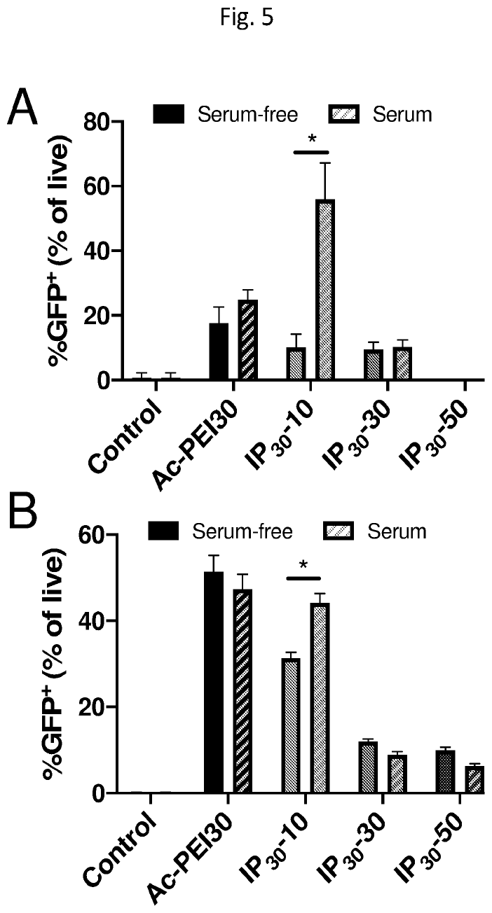

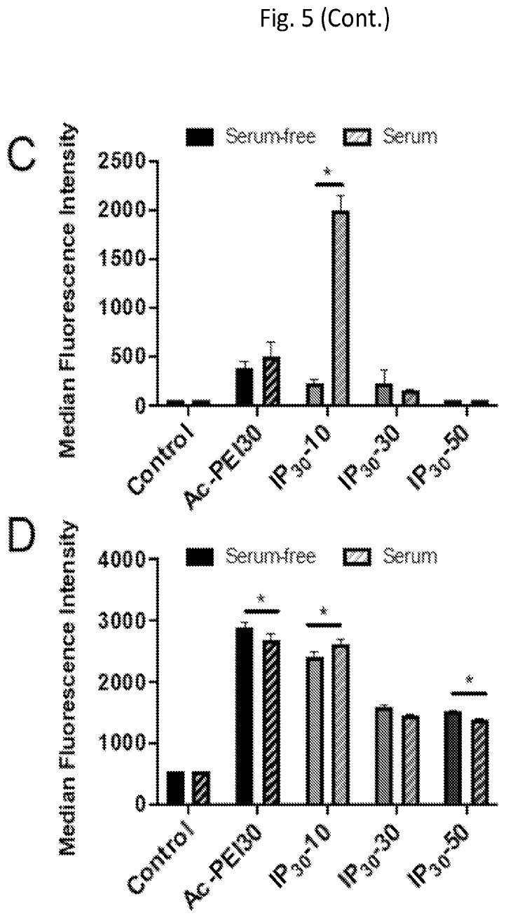

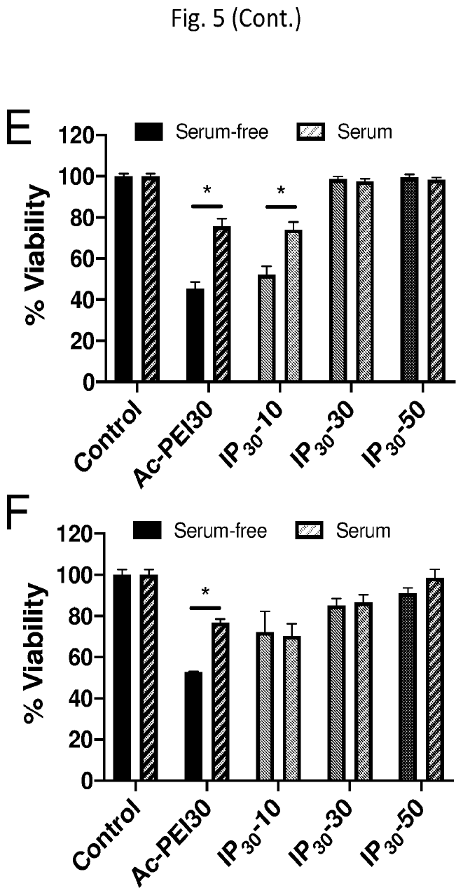

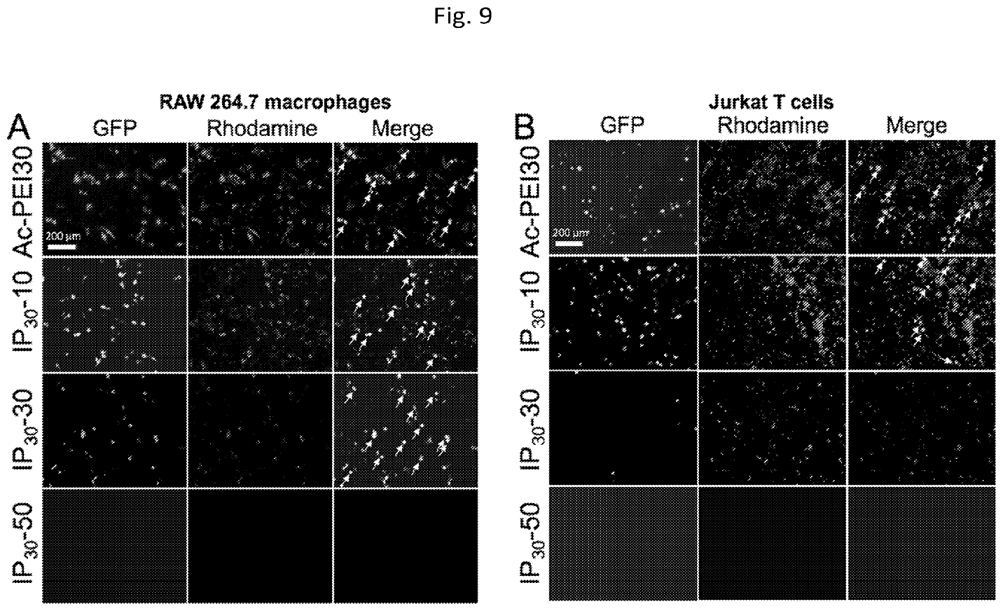

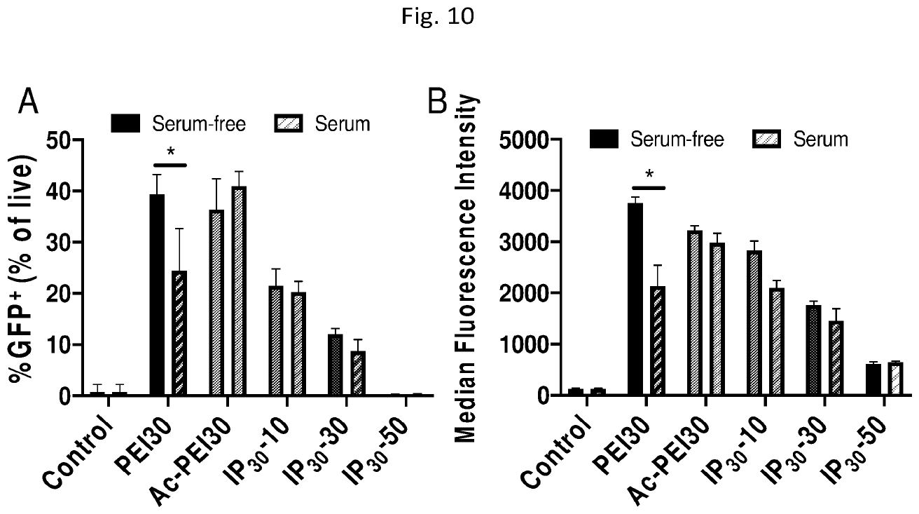

Flow cytometry was used to quantify differences in transfection efficiency for IPs in serum-containing or serum-free medium. The impact of PEMA enveloping on transfection efficiency and toxicity of IPs in RAW 264.7 and Jurkat cells is shown in FIG. 5. Transfection of RAW 264.7 cells (FIG. 5A) was less effective compared to Jurkat cells (FIG. 5B) in serum-free conditions, where all IPs tested resulted in less than 10% of cells GFP⁺. Interestingly, a 225% enhancement was measured in RAW 264.7 cells in serum-containing medium for IP₃₀-10 compared to Ac-PEI30. However, this difference was not observed for Jurkat cells, where IP₃₀-10 was not significantly different from Ac-PEI30 at similar conditions. The ability for IPs to transfect either cell type efficiently was lost using IP₃₀-30 and IP₃₀-50. A potential reason for the loss in transfection efficiency for IP₃₀-30 is the increased size of the complex due to aggregation in this condition (FIG. 4B). The reduced transfection efficiency of IP₃₀-50 was attributed to a highly negative surface charge that reduced nano-bio interactions as supported by FIG. 9. The MFI for GFP expression was assessed for RAW 264.7 and Jurkat cells in FIGS. 5C and D, respectively. Notably, in accordance with the % GFP⁺ enhancement for IP₃₀-10 in RAW 264.7 cells, a 400% increase in MFI was measured over Ac-PEI30. The MFI for Jurkat cells followed a similar trend as % GFP⁺. For both cell types, the viability was increased with increasing percentages of PEMA enveloping and in the presence of serum (greater than 10% PEMA) (FIGS. 5E and F). A similar trend for % GFP⁺ enhancement and MFI for DC2.4 cells was observed as Jurkat cells except that Ac-PEI30 treatment resulted in greater expression than IP₃₀-10 (FIG. 10). These results demonstrated that IPs prepared at high N/P ratios significantly increased the transfection efficiency of RAW 264.7 cells and retained similar effectiveness of Ac-PEI30 for Jurkat and DC2.4 cells. Importantly, the reduced non-specific cellular interactions for IPs such as IP₃₀-50 may offer an avenue for further development of the IP platform for specific cell targeting through incorporation of targeting ligands (Smith et al., Nat Nanotechnol 2017, 12, 813-820; Pearson et al., ACS Nano 2016, 10, 6905-6914.).

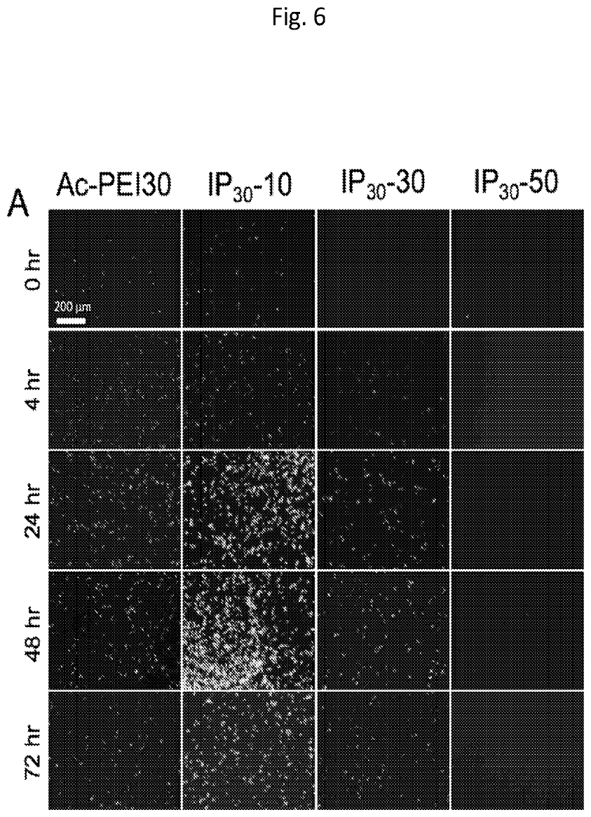

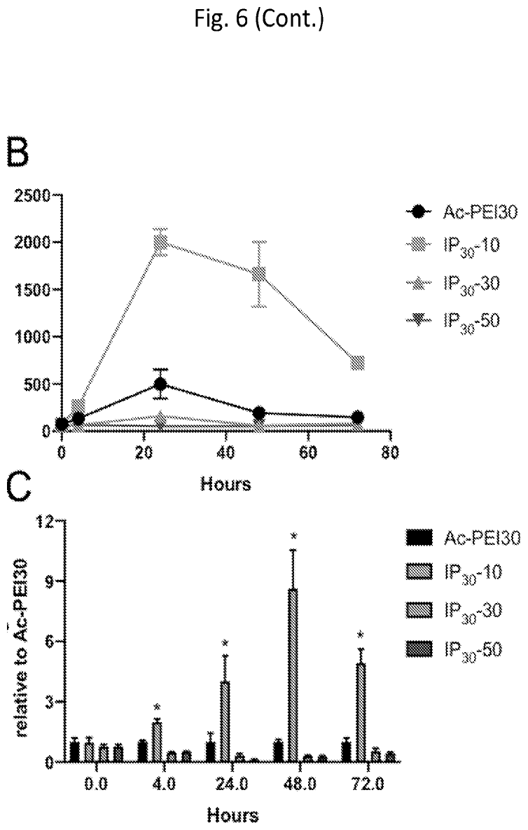

Controlling GFP expression in vitro: To further understand the persistence of GFP expression and the role that PEMA enveloping of polyplexes plays in this process, we evaluated GFP expression using a combination of fluorescence microscopy and flow cytometry at 5 timepoints using RAW 264.7 cells. FIGS. 6A and 6B show the significant enhancements in GFP expression in cells from 24-72 h in serum-containing medium. FIG. 6C shows the fold-change MFI relative to Ac-PEI30 for each of the IPs evaluated. The most significant enhancements were found for IP₃₀-10 at all timepoints longer than 4 h (2-fold). The enhancements further increased to 4-fold at 24 h and 8-fold at 48 h before slightly decreasing to 5-fold at 72 h. A noteworthy finding from this study was that a significant increase in GFP expression after 4 h was measured for IP₃₀-10 indicating that the PEMA enveloping potentially enhanced the unpackaging of plasmid DNA from Ac-PEI. This enhancement in unpackaging is likely due to three factors: 1) Ac-PEI has a greater capacity to release DNA due to its acetylation (Forres et al., Pharm Res 2004, 21, 365-71; Gabrielson et al., Biomacromolecules 2006, 7, 2427-2435.); 2) Ac-PEI has a lower pKₐ than PEI, which affects endosomal buffering capacity; and 3) PEMA improves the dissociation of DNA from Ac-PEI by dissociating from the surface of polyplexes at lysosomal pH (4.5-5) (PEMA: pKa₁=4.16; pKa₂=5.61) (Johnson et al., Durham University, 2010) that leads to endosomal membrane destabilization and improves cytosolic delivery of DNA (Evans et al., Nat Commun 2019, 10, 5012). Therefore, the type of anionic polyelectrolyte enveloping the polyplex potentially plays a significant role in controlling the protein expression in cells which could be very important design criteria to leverage in for future IP designs.

### CONCLUSIONS

Engineering of immune cells through delivery of nucleic acids has the widespread potential to correct or functionally reprogram genetic aberrations present in diseases or as therapeutics in the case of cancer, autoimmune diseases, or others (Weber et al., Cell 2020, 181, 46-62). The presence of serum has been demonstrated as a hindrance to achieve high transfection efficiencies and in situ applications of gene delivery will require delivery platforms to be effective in complex medium such as blood. Here, we developed a simple and effective, serum-independent platform technology, immunoplexes (IPs), consisting of Ac-PEI enveloped with an anionic polyelectrolyte (PEMA) to enhance the transfection of plasmid DNA into innate and adaptive immune cells. We identified through modulation of the N/P ratio that the negative impact of serum in lymphocyte transfection could be overcome and that the high N/P ratio used for the formulation of IPs was critical to ensure serum stability. Furthermore, the incorporation of PEMA enabled controllable cellular interactions and transfection efficiencies to be engineered, improved IP unpackaging, and showed a beneficial toxicity profile. The optimized IP₃₀-10 formulation resulted in an increased duration of gene expression compared to other Ac-PEI30 and other IP variants with a maximum 8-fold enhancement in gene expression over 3 days. Finally, we foresee that formulations such as IP₃₀-50, which showed limited cellular interactions, transfection efficiency, and low toxicity will be useful when developed further for in situ targeted gene delivery applications as PEMA can be functionalized with targeting ligands directed toward cell types of interest as similarly achieved using other anionic polymers (Smith et al., Nat Nanotechnol 2017, 12, 813-820; Zhao et al., Cell 2020, 181, 151-167). Our future studies aim to address these potential factors and applications to improve the efficiency of IPs as a non-viral gene delivery platform for targeted modulation of immune-mediated diseases.

### Example 2. Targeted Non-Viral Gene Delivery Systems for Effective Reprogramming of Immune Responses to Overcome Immunosuppression

Background and Significance: Targeting and reprogramming of immune cell populations in vivo using nanoparticles has the potential to restore innate and adaptive immune cell deficits that impair recovery from infection and survival over the long term. Delivery of nucleic acids to immune cells has widespread potential to correct genetic aberrations present in a variety of diseases. In many cases, the introduction of genetic materials into cells relies heavily on physical or viral approaches. Physical methods, including electroporation and microinjection show high transfection efficiency yet suffer from high toxicity and are not amenable to cell-specific targeting, which hinders their in vivo implementation. Viral vectors are highly efficient, but applications are limited due to unwanted genetic mutations and immunogenicity, posing concerns about safety in clinical translation. In comparison to that, non-viral gene delivery methods have the potential to overcome many of these limitations, particularly regarding toxicity and targetability in vivo. Here, we have prepared immunoplexes (IPs) comprised of acetylated polyethyleneimine (PEI-Ac)-plasmid DNA complexes that have been coated with negatively charged poly(ethylene-alt-maleic acid) (PEMA) to minimize non-specific interactions and toxicities with immune cells. IPs represent a modular gene delivery platform that has the potential to be functionalized with a variety of targeting ligands to achieve high levels of cell-specific transfection.

### Experimental Methods:

Materials: Acetic anhydride, branched polyethyleneimine (PEI, MW 25 KDa), trimethylamine, 1-Ethyl-3-(3-dimethylaminopropyl)carbodiimide (EDC) were purchased from sigma. N-hydroxysulfosuccinimide (Sulfo-NHS) was purchased from Fisher Scientific (Waltham, MA). Poly(ethylene-alt-maleic anhydride) (PEMA) was purchased from Polyscience, Inc. (Warrington, PA). Anti-CD3e F(ab′)₂ was purchased from Bioxcell (West Lebanon, NH), IL-2 was purchased from Peprotech (Rocky Hill, NJ).

Antibodies and Flow cytometry: All antibodies were purchased from BioLegend (San Diego, CA). Cell staining was conducted according to BioLegend protocols. Flow cytometric data were collected using a Becton Dickinson LSR II flow cytometer. Analysis was performed using FCS Express 6 (De Novo, Glendale, CA). FcR blocking was performed with anti-CD16/32 (Biolegend) prior to staining with various combinations of the following antibodies: anti-CD4 (RM4-5), -CD25 (PC61) (Biolegend). Viability was assessed with 4′,6-Diamidino-2-Phenylindole, Dilactate (DAPI) exclusion dye (Biolegend). anti-CD3, anti-CD28 were purchased from Thermo Fisher (Waltham, MA),

Cell culture: RAW 264.7 cells were cultured in Dulbecco's Modified Eagle Medium (DMEM) supplemented with penicillin (100 units/mL), streptomycin (100 μg/mL), and 10% heat-inactivated fetal bovine serum (FBS) (Invitrogen Corporation, Carlsbad, CA). Human Jurkat T lymphocyte cells were cultured in RPMI 1640 supplemented with 10% heat inactivated FBS and 1% penicillin/streptomycin at 37° C. and 5% CO₂ atmosphere. Primary CD4⁺ T cells were isolated from the spleen of C57BL/6J mice and purified using magnetic activated cell sorting. The spleen was macerated over a 40 μm cell strainer, centrifuged, and the RBCs were lysed using ACK lysis buffer. CD4⁺ T cells were cultured in a 96 well plate at 2×10⁵ cell/well with complete RPMI media supplemented with a cocktail of anti 2.5 μg/mL anti-CD3, 2 μg/mL anti-CD28 and 1 ng/mL IL-2.

Mice: Female C57BL/6J (6-8 weeks old) were purchased from The Jackson Laboratories (Bar Harbor, ME). The mice were housed under specific pathogen-free conditions in the University of Maryland Comparative Medicine facilities and all mice procedures and experiments were compliant to the protocols of the University of Maryland Animal Care and Use Committee.

Acetylation of PEI (PEI-Ac): 500 mg of PEI (0.02 mmol, 25000 g/mol) was dissolved in 10 mL methanol. Subsequently, various amounts of triethylamine (5 molar equivalents to acetic anhydride) was added to the stirring solution. Finally, acetic anhydride was then added to the mixture to achieve percentages of acetylation of 20%, 40%, 60% of the total primary amines. The reaction was allowed to stir overnight at room temperature and the modified PEI was purified by dialysis and recovered by lyophilization.

| Theoretical | | | |
|---|---|---|---|
| modification | PEI | Triethylamine | Acetic anhydride |
| 20% | 500 mg | (2.32 mmol AA * | (237.42 mg, 2.32 |
| | | 5 = 11.6 mmol, | mmol, 219.8 (μL) |
| | | 1.6 mL) | |
| 40% | 500 mg | 3.2 mL | 439.7 μL |
| 60% | 500 mg | 4.8 mL | 659.5 μL |

Synthesis of anti-CD3e F(ab)₂ conjugated poly(ethylene-alt-maleic anhydride) (PEMA-Ab): PEMA was dissolved in water at a concentration 20 mg/mL then NaOH was added to obtain pH 7.0. After that 1.53 mg EDC was dissolved in 1 mL of water and added to the PEMA solution and stirred for 15 minutes at room temperature. Next, 1.73 mg of sulfo-NHS was dissolved in 1 mL of water and added to the mixture and stirred for another 15 minutes. The resulting activated PEMA was conjugated with antibody by adding 4 nmole of anti-CD3e F(ab′)₂ to the solution the reaction was carried out overnight at 4° C. After the reaction the solution was dialyzed to remove excess reagents and recovered by lyophilization.

Characterization of PEMA-Ab conjugate: PEMA-antibody conjugates, (PEMA-Ab) were characterized by molecular weight estimation using SDS polyacrylamide gel electrophoresis (SDS-PAGE, 4-15%, non-reducing conditions, 75 mV, 1-2 hr) and visualized by coomassie blue staining.

GFP Plasmid preparation: Plasmids were prepared by following a standard molecular biology protocol. DH5a Competent Cells were transformed with eGFP plasmid (courtesy of National Center for Toxicological Research, FDA, Jefferson, AR), which encoded for kanamycin resistance. A single colony was grown up in an overnight culture, lysed and purified using the Qiagen Maxiprep Endotoxin Free kit. The purity have been checked by measuring absorbance at 260 nm and 280 nm and by running agarose gel electrophoresis. The concentration of DNA was determined by Nanodrop and stored at −20° C.

Optimization of PEI-Ac polyplex formation: Fresh polyplex solutions were prepared before transfection. Polyplexes formed from PEI-Ac and pDNA were initially prepared at N/P ratios ranging from 0 to 20. First, plasmid DNA was diluted in HEPES (20 mM, pH 7) buffer at a concentration 500 ng/mL. A fresh solution of PEI-Ac was prepared at a concentration of 1 mg/mL. Next appropriate amount of PEI-Ac and plasmid DNA were mixed together in a microcentrifuge tube and incubated for 10 minutes. We prepared 6 PEI-Ac:GFP polyplexes for each of the three types of PEI-Ac. The stability of polyplex formation was assessed using DNA agarose (1%) gel electrophoresis. The plasmid DNA was visualized by ethidium bromide.

Preparation of immunoplexes (IPs) and targeted immunoplexes (tIPs): Fresh polyplex solutions were prepared before transfection. Polyplexes formed from PEI-Ac and pDNA were initially prepared at N/P ratios ranging from 0 to 20. Here, we focused on N/P ratio 20 and 40 to facilitate high stability of the polyplexes during the PEMA coating process. First, plasmid DNA was diluted in HEPES (20 mM, pH 7) buffer at a concentration 500 ng/mL. A fresh solution of PEI-Ac, PEMA and PEMA-Ab was prepared at a concentration of 1 mg/mL. Next appropriate amount of PEI-Ac and plasmid DNA were mixed together in a microcentrifuge tube and incubated for 10 minutes. The appropriate amount of PEMA and PEMA-Ab was added to the mixture to make PEMA and PEMA-Ab coated polyplexes which we denote as immunoplexes (IPs) and targeted immunoplexes (tIPs), respectively. We prepared a library of formulations of IPs and tIPs by adding different wt % of PEMA and PEMA-Ab (10 wt %, 30 wt %, 50 wt %) respectively with respect to PEI-Ac.

Stability of IPs in Presence of PEMA and Serum: A solution of plasmid DNA was prepared in HEPES buffer (pH 7.0) at a concentration 500 ng/μL. Stock solution of PEI-Ac and PEMA solution is prepared at a concentration of 1 mg/mL. Next the appropriate amount of PEI and plasmid DNA were mixed to maintain N/P ratio 20 and 40 and kept at room temperature for 10-20 minutes. IPs were prepared by adding varying amount of PEMA from 10 wt % to 50 wt % with respect to PEI-Ac. The IPs were kept at room temperature for 15-30 minutes after which 6 μL of loading dye was added and run on a 1% agarose gel. The stability of those IPs in presence of serum was also determined by DNA gel retardation assay. First, polyplexes with N/P ratio 40 were prepared and coated with 10 wt %, 30 wt % and 50 wt % of PEMA. After that IPs were mixed with 10% and 55% of FBS and incubated for 15-30 minutes. Next 6 μL of loading dye was added and run on a 1% agarose gel. The plasmid DNA was visualized by ethidium bromide.

In vitro Transfection Study: Polyplexes, IPs, and tIPs were prepared with N/P ratio 20 and 40 containing 2 pg of plasmid DNA. 10-50 μL of these polyplexes were added to the cell culture medium and incubated for 4 hrs. RAW 264.7 cells are adherent cells and therefore the cells were washed with PBS and incubated with fresh DMEM overnight. In contrary, Jurkat cells are suspension cells and therefore after incubation with the polyplexes and IPs, the cells were collected in a microcentrifuge tube and centrifuged. Next the supernatant was removed and fresh RPMI was added to the cell pallet and made cell suspension which was put back into the well plate. The GFP expression was visualized using a fluorescence microscope 24 hr post transfection.

### Results and Discussion:

Acetylation of PEI (PEI-Ac): Acetylation of PEI can reduce the buffering capacity and that gene delivery efficiency is enhanced up to 21 fold after 43% acetylation of PEI. The primary and secondary amine groups of PEI polymer react with acetic anhydride in presence of triethylamine leading to acetylated PEI (PEI-Ac). NMR spectroscopy was used to determine the percent acetylation as previously described. The extent of acetylation was measured to be 25.5%, 40.4% and 54.5% (P1, P2, P3 respectively). The complexation stability of these polymers with GFP plasmid DNA have been verified by agarose gel retardation assay. Higher N/P ratio offered higher stability and better transfection, however it produces higher cellular toxicities, off target cellular interaction and cannot be considered to be a good transfecting agent for in vivo studies. Nonspecific therapeutic approach always causes aberrant activation or suppression of immune cells leading to increased risk of inflammation and malignancies. Therefore, strategies have been developed for specific cellular genetic reprogramming of immune cells. Here we are trying to modify this polyplexes with anionic charged polymer PEMA by layer-by-layer (LbL) assembly. This modification will help to minimize nonspecific interaction with cells and at the same time antibody attachment to the surface of the polyplex will enable specific cellular interactions with immune cells of interest. We sought to develop a strategy to deliver GFP plasmid DNA specifically to T lymphocytes. To target T lymphocytes, we used anti-CD3E F(ab′)₂ antibody fragment as previously reported. This antibody fragment was conjugated on the surface of PEMA (PEMA-Ab) using EDC/NHS carbodiimide chemistry. First, the carboxylic acid groups of PEMA were activated using EDC and NHS, then the activated carboxyl group was reacted with the amine groups of anti-CD3E F(ab′)₂. The PEMA-Ab coated polyplexes (tIPs) were designed to interact specifically with T cells by recognizing T cell receptor, facilitate internalization by receptor mediated endocytosis and deliver GFP plasmid DNA. The hydrodynamic diameter of the polyplexes (N/P 40) was approximately 240 nm with zeta potential of +25 mV. After surface modification with anionic polymer PEMA, surface charge of the polyplexes was significantly reduced. As the amount of PEMA was increased, the zeta potential starts decreasing and it was significantly lowered when 50 wt % PEMA is added. In contrary, the hydrodynamic size was increased as wt % of PEMA was increased which signifies the coating of PEMA on the surface of polyplexes. Despite the ability to wrap around polyplexes, PEMA can also compete the plasmid DNA from the polyplexes when a significant amount is added which leads to destabilize the polyplexes. To find out the effect of PEMA on IP stability, agarose gel electrophoresis was run. It was observed that up to 50 wt % of PEMA can maintain the stability of IPs, yet IPs became unstable as the PEMA concentration was further increased. Finally, the effect of serum was also verified in polyplexes stability. Incubation of IPs in the presence of physiological concentrations of serum (55%) did not lead to destabilization and release of the plasmid DNA. This provided evidence that the formulated polyplexes display beneficial physicochemical properties that may allow for their successful use in vivo.

In vitro Transfection in RAW 264.7 and Jurkat cells: The transfection efficiency of polyplexes was evaluated in vitro using Raw264.7 and Jurkat cells. IPs with variable coating with PEMA (10 wt %, 30 wt % and 50 wt %) were incubated with cells for 4 hrs. After that the cells were washed and incubated with fresh complete media for overnight and imaged using fluorescence microscopy. We had also prepared rhodamine conjugated PEI-Ac (Rh-PEI-Ac) to confirm the uptake of the polyplexes. Significant difference in transfection and GFP expressions were noted for RAW264.7 cells between serum-containing and serum-free conditions. The transfection efficiency in RAW 264.7 cells was increased in presence of serum which was more relevant to the physiological conditions and could be very useful for improved gene transfection. The uptake of polyplexes and the transfection efficiency was measured to be reduced with increased amount of PEMA in both RAW264.7 and Jurkat cells which clearly suggest that PEMA coating can reduce non-specific cellular interactions.

Primary T cell transfection: Primary CD4⁺ T cells were isolated from the spleen of C57BL/6J mice using magnetic activated cell sorting. The T cells were cultured 24 hrs in a 96 well plate coated with anti-CD₃ at 2×10⁵ cells/well in complete RPMI 1640 media supplemented with 2 μg/mL anti-CD28 and 10 ng/mL IL-2. Next the cells were incubated with polyplexes, IPs and tIPs (N/P 40) for 4 hrs. After that the cells were centrifuged to remove the polyplex and suspended in fresh RPMI 1640 media supplemented with anti-CD28 and IL-2 and incubated for 24 hrs. Next the cells were collected in a tube and measured the GFP expression by flow cytometry. It is clearly observed that tIPs are able to transfect T cells compared to non-targeted IPs (65.1% versus 1.6%).

### Example 3. Design and Synthesis of Functional Polyesters as Efficient Gene Delivery Carrier

Materials: 1) N-Boc Serinol, Sebacoyl chloride, Pyridine, Trifluoroacetic acid, N methyl diethanolamine, anhydrous dichloromethane (DCM, >99.8%), methanol (>99.9%) and D2O were purchased from Sigma. dimethylsulfoxide-d6 was purchased from Cambridge isotope labs Inc.

### Synthesis Procedure:

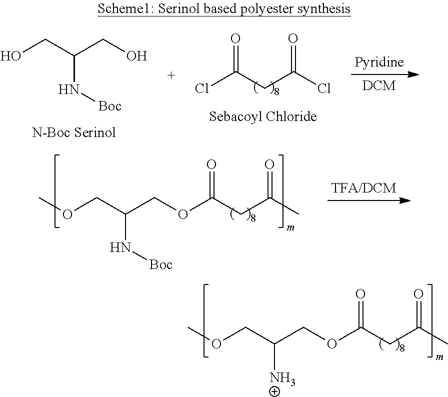

Serinol based polyester by polymerization between N-Boc serinol and sebacoyl chloride in presence of pyridine as HCl scavenger. At first 956.1 mg of N-Boc serinol (5 mmol) was dissolved in 5 ml DCM and added in a 50 ml round bottom flask. After that 808.79 μl (10 mmol) of pyridine was added to it and stirred under Ar atmosphere at 500 rpm for 15 minutes. Next 1 ml of sebacoyl chloride (5 mmol) was dissolved in 5 ml of DCM and slowly added to the reaction mixture for 30 minutes. After complete addition, the reaction was further carried out for 12 hrs. The white precipitate was removed by centrifugation. The supernatant containing DCM was removed in rotary evaporator. Next the resulting solid product was dissolved in minimum amount of methanol and the polymer was purified by precipitation into distilled water. After that the product was dried in air for two days and 1HNMR spectra was measured. Next the deprotection of primary amine was performed. 500 mg of pure polyester was dissolved in 1 ml DCM and 1 ml of TFA was added to it. The reaction mixture was stirred for 4 hrs to perform complete deprotection of the primary amine. After that the DCM and TFA was removed by rotary evaporator. The deprotection of primary amine was analyzed by 1HNMR.

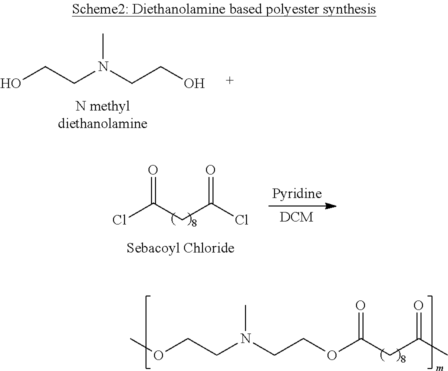

N methyl diethanolamine based polyester containing tertiary amine was prepared via polymerization between N methyl diethanolamine and sebacoyl chloride. First, 574 μl of N methayl diethanolamine and 808.79 μl of pyridine were dissolved in 10 ml DCM and added in a 50 ml round bottom flask. The mixture was stirred at 500 rpm in argon atmosphere for 15 minutes. Next, 1.067 ml sebacoyl chloride was dissolved in 5 ml DCM and added to the mixture dropwise over 30 minutes. After complete addition, the reaction was carried out for overnight (>12 hrs) for complete consumption of the starting materials. Next, DCM was evaporated in vacuum and the white solid product was obtained. The resulting solid was dissolved in methanol and the polymer was purified by precipitation by adding diethyl ether. The precipitated product was isolated by centrifugation and dried in vacuum. The polymer was characterized by ¹HNMR after dissolving in D₂O.

### Results and Discussion:

Polymer Synthesis and ¹HNMR characterization: Primary amine containing polyesters were prepared by polymerization of N-boc serinol and sebacoyl chloride at a 1:1 mole ratio in presence of pyridine. Primary amine group of serinol can interfere with the polymerization, therefore Boc protected serinol was used as a starting material. Pyridine acts as catalyst by extracting HCl produced during the reaction and precipitate as a salt in DCM. After the reaction, the pyridine salt was removed by centrifugation and DCM in the supernatant was removed by vacuum evaporation. The resulting solid product was dissolved in methanol and added in distilled water to get the polymer as precipitate. The precipitated product was dried in air for 2 days. The polymer backbone contains Boc protected primary amines which needs to be removed for further functionalization. To remove the Boc protection TFA was added to the polymer solution. The reaction was carried out for 4 hrs to make sure complete deprotection. ¹HNMR spectra confirms the polymerization of the monomers and complete deprotection of primary amine groups. The signals around 1.402 ppm represents the -Boc protecting group which was disappeared after the treatment with TFA. This signifies the complete deprotection of primary amine group from the polymer backbone.

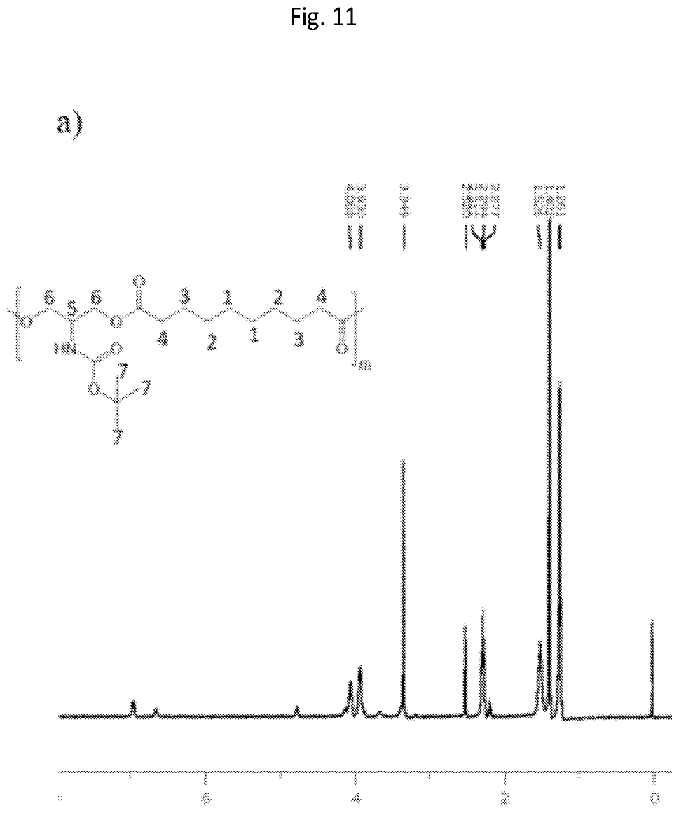

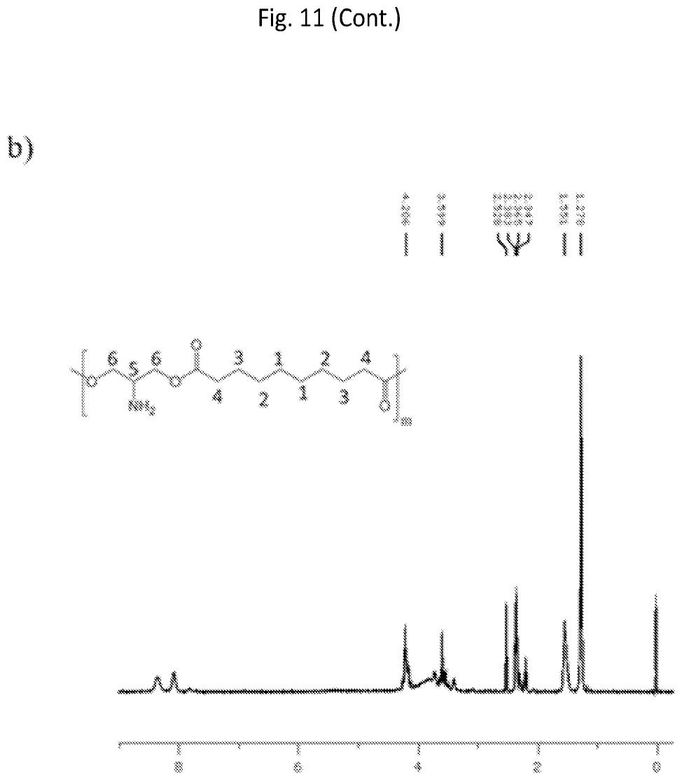

¹HNMR: (400 MHz, DMSO-d⁶): δ (ppm): 1.278 (¹CH₂ ²CH₂), 1.402 (⁷CH₃), 1.526 (³CH₂), 2.2-2.3 (⁴CH₂), 3.9-4.0 (⁵CH₂, ⁶CH₂) (FIG. 11)

We also prepared N-methyl diethanolamine based polyester which offers tertiary amine in the polymer backbone. Tertiary amine can offer higher positive charge and higher endosomal escape efficiency than primary amines after internalization into intracellular compartment. The polymer was synthesized by reacting N-methyl diethanolamine and sebacoyl chloride at a 1:1 mole ratio. Pyridine was used as a catalyst for HCl scavenger. The reaction was carried out similarly as above. After completion of the reaction DCM was removed by vacuum evaporation. The solid product thus obtained was dissolved in methanol and the polymer was precipitated by adding diethyl ether. The resulting polymer was dried and characterized by ¹HNMR.

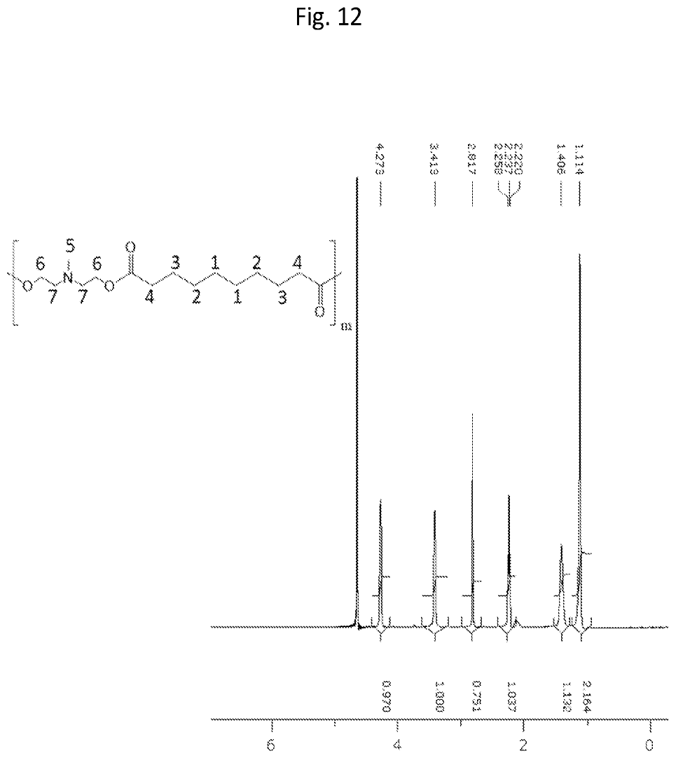

¹HNMR: (400 MHz, D₂O): δ (ppm): 1.1 (¹CH₂, ²CH₂), 1.406 (³CH₂), 2.220-2.258 (⁴CH₂), 2.817 (⁵CH₃), 3.413 (⁷CH₂), 4.273 (⁷CH₂). (FIG. 12)

Preparation of polyplexes: 10 mg of poly serinol and poly diethanolamine were dissolved in 1 ml DMSO and 1 ml H₂O, respectively. After that polyplexes were prepared at different weight ratio ranging from 100:1 to 600:1 (polymer:plasmid) containing 2 μg of plasmid. The appropriate amount of polymer and GFP plasmid were mixed together and incubate for 10 minutes to obtain the polyplexes. Polyplexes prepared from polyserinol and poly diethanolamine are termed as Polyplex^{serinol} and Polyplex diethanolamine respectively.

Hydrodynamic size and zeta potential measurement: polyplex solutions were prepared with 2 pg of GFP plasmid in HEPES buffer. One milliliter of each sample was added into a disposable cuvette, and the size was measured using a Zetasizer Nano ZS (Malvern Instruments Inc.). Fifteen runs were performed in triplicate for each sample. Subsequently, samples were transferred to a folded capillary cell (Malvern) and zeta-potential measurements were performed in triplicate for each sample using the Zetasizer Nano ZS (Malvern).

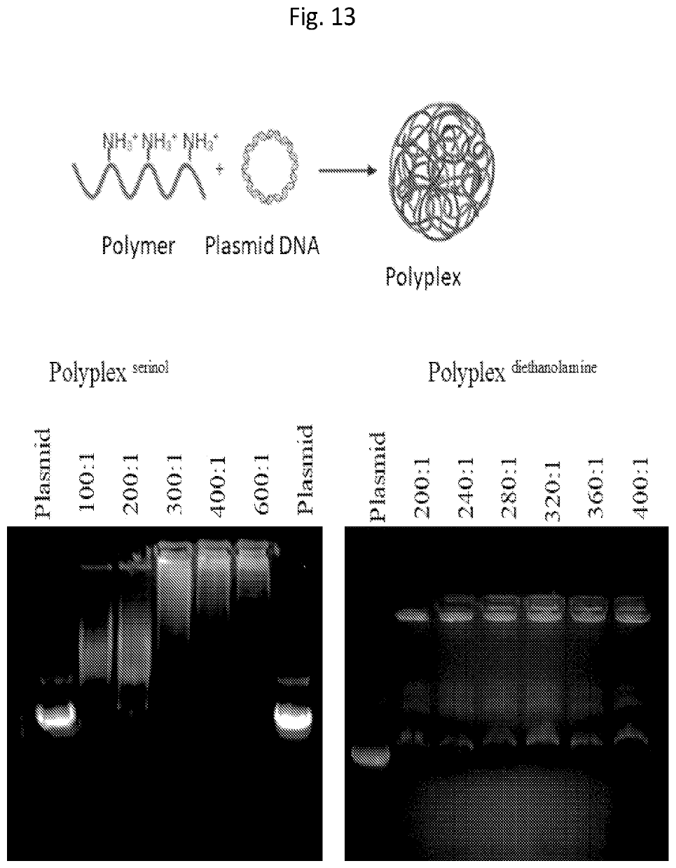

Stability of polyplexes. The stability of polyplexes is critical to ensure pDNA can be efficiently delivered to immune cells of interest both in vitro and in vivo without destabilization. The stability of the polyplexes was assessed by DNA gel electrophoresis (FIG. 13). It was observed that Polyplex^{diethanolamine} offered higher stability of the polyplexes compared to Polyplex^{serinol}. This was also obvious from the zeta potential of the two polyplexes (Table 2). Polyplex^{diethanolamine} showed higher positive charge than Polyplex^{serinol} and therefore the former provides higher stability to the polyplexes.

TABLE 2
Size and Zeta potential of two different polyplexes

| | Size, PDI | Zeta (mV) |
|---|---|---|
| Polyplex^{serinol} | 450.1, 0.063 | −13.6 |
| Polyplex^{diethanolamine} | 115.1, 0.184 | +25.4 |

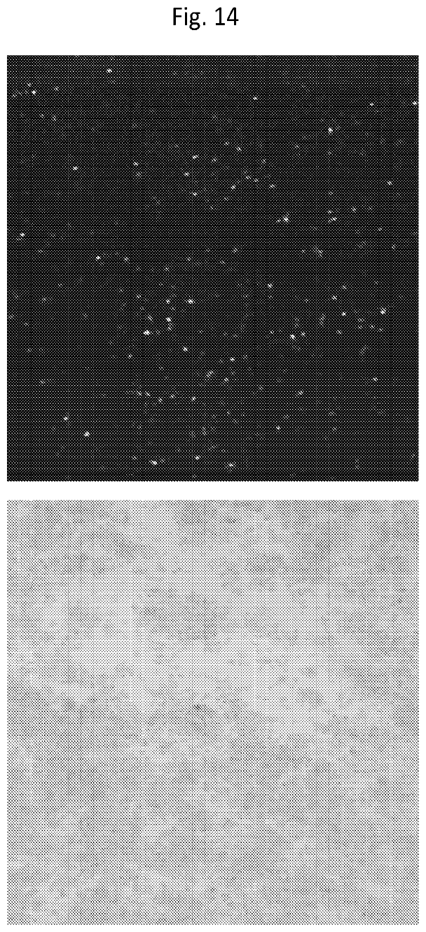

In Vitro Transfection of RAW 264.7 Macrophages: Polyplexes were prepared with polyethanolamine and 2 μg of pDNA encoding GFP (Ac-PEI30). RAW 264.7 cells were treated with polyplexes for 4 h in serum-containing medium followed by washing to remove excess complex prior to overnight incubation in complete media. GFP expression was visualized using a revolve fluorescence microscope (ECHO, San Diego, CA) at 24 h post-transfection. See FIG. 14.

1. A non-viral polyplex particle for delivering nucleic acid to cells, comprising
i) an effective amount of polyethylenimine complexed with an effective amount of the nucleic acid; and
ii) an effective amount of an anionic biomaterial that envelops the complexed polyethylenimine and nucleic acid.

2. The non-viral polyplex particle of claim 1, wherein the polyethylenimine is acetylated.

3. The non-viral polyplex particle of claim 1, wherein the polyethylenimine is branched.

4. The non-viral polyplex particle of claim 1, wherein the non-viral polyplex particle has an N/P ratio of at least 7.5.

5. The non-viral polyplex particle of claim 1, wherein the non-viral polyplex particle has an N/P ratio of at least 15.

6. The non-viral polyplex particle of claim 1, wherein the non-viral polyplex particle has an N/P ratio of at least 30.

7. The non-viral polyplex particle of claim 1, wherein the anionic biomaterial is selected from the group consisting of poly(glutamic acid), poly(aspartic acid), poly(acrylic acid), hyaluronic acid, poly(methyl vinyl ether-alt-maleic acid), poly(isobutylene-alt-maleic acid), poly(ethylene-alt-maleic acid), poly(ethylene-altmaleic anhydride), and combinations thereof.

8. The non-viral polyplex particle of claim 1, wherein the anionic biomaterial is poly(ethylene-alt-maleic acid).

9. The non-viral polyplex particle of claim 1, wherein the percentage acetylation of polyethylenimine is from about 10% to about 60%.

10. The non-viral polyplex particle of claim 1, wherein the percentage acetylation of polyethylenimine is from about 20% to about 50%.

11. The non-viral polyplex particle of claim 1, wherein the percentage acetylation of polyethylenimine is from about 20% to about 25%.

12. The non-viral polyplex particle of claim 1, wherein the percentage acetylation of polyethylenimine is about 25%.

13. The non-viral polyplex particle of any of claims 1-12 claim 1, wherein the anionic biomaterial is present at a weight percent of about 0.1% to about 67%.

14. The non-viral polyplex particle of claim 1, wherein the anionic biomaterial is present at a weight percent of about 5% to about 35%.

15.-17. (canceled)

18. The non-viral polyplex particle of claim 1, wherein the polyplex further comprises a targeting moiety that enables delivery of the nucleic acids to a target cell, wherein the targeting moiety binds to the surface of the target cell, wherein the non-viral polyplex particle is internalized by the target cell by receptor mediated endocytosis.

19. The non-viral polyplex particle of claim 18, wherein the targeting moiety is selected from the group consisting of a protein, a cell adhesion molecule, an antibody, a peptide, a sugar, a small molecule, and any combination thereof.

20. The non-viral polyplex particle of claim 18, wherein the targeting moiety comprises a single chain antibody.

21. The non-viral polyplex particle of claim 18, wherein the targeting moiety comprises a single chain (scFv) variable fragment antibody.

22. The non-viral polyplex particle of claim 18, wherein the target cell is an immune cell.

23.-26. (canceled)

27. A non-viral polyplex particle for delivering a nucleic acid to cells, comprising
i) an effective amount of an amine containing cationic biomaterial complexed with an effective amount of the nucleic acid; and
ii) an effective amount of an anionic biomaterial that envelops the complex of amine containing cationic biomaterial and nucleic acid, wherein the non-viral polyplex particle has an N/P ratio of at least 15.

28. The non-viral polyplex particle of claim 27, wherein the non-viral polyplex particle has an N/P ratio of at least 30.

29. The non-viral polyplex particle of claim 27, wherein the amine containing cationic biomaterial is selected from the group consisting of polylysine, polyethylenimine, chitosan, polyamidoamine dendrimers, polyhistine, poly(betaamino esters), poly(2-dimethylaminoethyl methacrylate), poly(2-[(2-aminoethyl)amino] ethyl aspartamide), poly(2-aminoethyl ethylene phosphate), spermine, spermidine, a cationic lipid and combinations thereof.

30. The non-viral polyplex particle of claim 29, wherein the cationic lipid is selected from the group consisting of DOPE, DOTAP, DOGS, DOSPER and combinations thereof.

31. The non-viral polyplex particle of claim 27, wherein the anionic biomaterial is selected from the group consisting of poly(glutamic acid), poly(aspartic acid), poly(acrylic acid), hyaluronic acid, poly(methyl vinyl ether-alt-maleic acid), poly(isobutylene-alt-maleic acid), poly(ethylene-alt-maleic acid), poly(ethylene-altmaleic anhydride), and combinations thereof.

32. The non-viral polyplex particle of claim 27, wherein the anionic biomaterial is poly(ethylene-alt-maleic acid).

33. The non-viral polyplex particle of claim 27, wherein the amine containing cationic biomaterial is polyethylenimine.

34. (canceled)

35. The non-viral polyplex particle of claim 33, the polyethylenimine is acetylated.

36.-52. (canceled)

53. A method of delivering a nucleic acid to a target cell, comprising administering an effective amount of the non-viral polyplex particle of claim 1 to the target cell.

54. A method for delivery of a nucleic acid to a target cell in a subject comprising administering an effective amount of the non-viral polyplex particle of claim 1.

55. (canceled)

56. (canceled)
